(19) United States
(12) Patent Application Publication
(10) Pub. No.: US 2026/0224795 A1
(43) Pub. Date: Aug. 6, 2026

(54) SURGICAL DRAIN STRIPPER DEVICE

(71) Applicant: Jose Arturo Bonilla, San Antonio, TX (US)

(72) Inventor: Jose Arturo Bonilla, San Antonio, TX (US)

(21) Appl. No.: 19/047,585

(22) Filed: Feb. 6, 2025

(51) Int. Cl. A61M 1/00

(52) U.S. Cl. CPC A61M 1/84; A61M 2209/04

(57) ABSTRACT

Apparatus and methods of manufacture for a device for stripping drainage tubes are disclosed. Such an apparatus can include a first arm comprising a first end and a second end, a first compression mechanism, mechanically coupled to the first arm, a second arm comprising a first end and a second end, and a second compression mechanism, mechanically coupled to the second arm.

### FIELD OF THE INVENTION

The present disclosure relates to medical devices, and more particularly, a medical device for clearing a surgical drain.

### BACKGROUND

Surgical drains are commonly employed in post-operative care to remove fluids and/or air from a patient's body, reducing the risk of infection and promoting healing. These drains often collect bodily fluids such as blood, pus, or lymph into a reservoir via a flexible tubing system. However, during the recovery process, the accumulation of impediments such as clotted material or viscous fluid within the tubing can obstruct the drain's functionality. Clearing such obstructions allows such drains to maintain the efficacy of the drainage system and ensure optimal patient outcomes.

Conventional methods for clearing obstructions in surgical drains involve approaches such as the manual manipulation (e.g., by squeezing, rolling, or “stripping” the tube by hand). While effective to some extent, these techniques often require considerable effort, may cause discomfort to the patient, and can be inconsistent in achieving thorough clearing. Additionally, manual stripping poses a risk of dislodging or damaging the tubing, leading to leaks, tears, or reduced drain integrity, particularly when performed by the patient or other medically untrained individuals.

Existing solutions have explored the use of clamp-based devices or mechanical tools to assist with clearing blockages. While these approaches may reduce some manual effort, they are cumbersome to use, require significant training, or fail to adapt to various drain configurations. Furthermore, the design of these devices result in devices that are flimsy and lack precise control, increasing the risk of excessive force that tends to compromise/dislodge the tubing and/or create additional complications (e.g., dislodging stitches at the surgical site, as well as the introduction of contaminants/disease the aforementioned damage can result in). These limitations highlight the need for an improved mechanism to safely and efficiently clear obstructions from surgical drains.

Accordingly, there remains a need for a surgical drain stripper that is ergonomic, reliable, and compatible with a wide range of tubing designs. Such a device should minimize patient discomfort, reduce the risk of tubing damage, and provide consistent performance in clearing blockages, even when performed by untrained individuals. Preferably, the design of such a device would readily facilitate production of the device (e.g., as by three-dimensional (3D) printing), in order to provide enhanced usability and effectiveness when maintaining surgical drains.

### BRIEF DESCRIPTION OF THE DRAWINGS

Embodiments of methods and systems such as those disclosed herein may be better understood, and its numerous objects, features, and advantages made apparent to those skilled in the art by referencing the accompanying drawings.

FIG. 1A is a simplified diagram illustrating a perspective view of a surgical drain stripper device having a flexion area at an lower portion of a flexible coupling, according to embodiments of methods and systems such as those disclosed herein.

FIG. 1B is a simplified diagram illustrating an end-on view of one end of the surgical drain stripper device shown in FIG. 1A, according to embodiments of methods and systems such as those disclosed herein.

FIG. 1C is a simplified diagram illustrating an end-on view of the surgical drain stripper device shown in FIG. 1A at an end opposite to that shown in FIG. 1C, according to embodiments of methods and systems such as those disclosed herein.

FIG. 2 is a simplified diagram illustrating a perspective view of a surgical drain stripper device having a flexion area at an upper portion of a flexible coupling, according to embodiments of methods and systems such as those disclosed herein.

FIG. 3 is a simplified diagram illustrating a perspective view of a surgical drain stripper device employing low-friction materials rather than rollers, according to embodiments of methods and systems such as those disclosed herein.

FIG. 4A is a simplified diagram illustrating a perspective view of a surgical drain stripper device that includes a gripper area as part of the roller shrouds, according to embodiments of methods and systems such as those disclosed herein.

FIG. 4B is a simplified diagram illustrating a perspective view of the surgical drain stripper device depicted in FIG. 4A, illustrating the motion involved in compressing a surgical drain tube passing therethrough, according to embodiments of methods and systems such as those disclosed herein.

FIG. 5A is a simplified diagram illustrating a perspective view of another surgical drain stripper device that includes a gripper area towards the end of each arm and using the lower arm as a stop for the upper arm, according to embodiments of methods and systems such as those disclosed herein.

FIG. 5B is a simplified diagram illustrating a side view of the surgical drain stripper device depicted in FIG. 5A illustrating the passage of a surgical drain tube therethrough, according to embodiments of methods and systems such as those disclosed herein.

FIG. 5C is a simplified diagram illustrating a side view of the surgical drain stripper device of the design depicted in FIG. 5A illustrating the passage of a surgical drain tube therethrough, in which a combination of a roller and a slide block is shown, according to embodiments of methods and systems such as those disclosed herein.

FIG. 6 is a simplified diagram illustrating a perspective view of yet another surgical drain stripper device that includes arm stops on the lower arm to limit motion of the upper arm, according to embodiments of methods and systems such as those disclosed herein.

FIG. 7 is a simplified diagram illustrating a perspective view of yet another surgical drain stripper device that includes insertion gaps, which allow for the installation of the surgical drain stripper device on a drain tube from the side, according to embodiments of methods and systems such as those disclosed herein.

FIG. 8 is a simplified diagram illustrating an overhead cutaway view of a surgical drain stripper device illustrating a roller design, according to embodiments of methods and systems such as those disclosed herein.

FIG. 9A is a simplified diagram illustrating an overhead cutaway view of a surgical drain stripper device illustrating another roller design, according to embodiments of methods and systems such as those disclosed herein.

FIG. 9B is a simplified diagram illustrating an overhead cutaway view of a surgical drain stripper device illustrating yet another roller design, according to embodiments of methods and systems such as those disclosed herein.

FIG. 10 is a simplified diagram illustrating an overhead cutaway view of a surgical drain stripper device illustrating still another roller design, according to embodiments of methods and systems such as those disclosed herein.

FIG. 11A is a simplified diagram illustrating an side view of a surgical drain stripper device that illustrates an embodiment in which the arms are separate components, according to embodiments of methods and systems such as those disclosed herein.

FIG. 11B is a simplified diagram illustrating an overhead cutaway view of a surgical drain stripper device such as that depicted in FIG. 11A, according to embodiments of methods and systems such as those disclosed herein.

FIG. 12 is a simplified diagram illustrating an side view of still another surgical drain stripper device, according to embodiments of methods and systems such as those disclosed herein.

FIG. 13 is a flow diagram illustrating an example of a stripper device manufacturing process, according to embodiments of methods and systems such as those disclosed herein.

FIG. 14 is a flow diagram illustrating an example of another stripper device manufacturing process, according to embodiments of methods and systems such as those disclosed herein.

FIG. 15 is a block diagram depicting a computer system suitable for implementing embodiments of methods and systems such as those disclosed herein.

FIG. 16 is a block diagram depicting a network architecture suitable for implementing embodiments of methods and systems such as those disclosed herein.

While the present disclosure is susceptible to various modifications and alternative forms, specific embodiments of the present disclosure are provided as examples in the drawings and detailed description. It should be understood that the drawings and detailed description are not intended to limit the present disclosure to the particular form disclosed. Instead, the intention is to cover all modifications, equivalents, and alternatives falling within the spirit and scope of the present disclosure as defined by the appended claims.

### DETAILED DESCRIPTION

While the methods and systems described herein are susceptible to various modifications and alternative forms, specific embodiments are provided as examples in the drawings and detailed description. It should be understood that the drawings and detailed description are not intended to limit such disclosure to the particular form disclosed. Instead, the intention is to cover all modifications, equivalents and alternatives falling within the spirit and scope of the appended claims.

### INTRODUCTION

The present disclosure relates to medical devices for clearing a surgical drains, as well as methods for manufacturing such apparatus. To accomplish such objectives, embodiments such as those described herein provide a drain tube stripping device that safely, effectively, and reliably clears clots, debris, viscous fluids, and/or other such impediments to fluid flow through the lumen of such drain tubes.

As briefly noted earlier, a surgical drain tube can be used in a variety of scenarios, and facilitates effective fluid management and promotes postoperative recovery in a variety of surgical procedures. Following extensive tissue manipulation, as commonly encountered in a variety of procedures, including general surgery, plastic surgery, orthopedic surgery, and thoracic surgery, among other types of surgery, fluid accumulation can occur. This fluid, which may consist of blood, serum, or lymph, can impede the healing process and increase the risk of complications like seroma formation.

The placement of surgical drain tubes allows for the controlled evacuation of these fluids. By effectively removing excess fluid, these devices mitigate the risk of hematoma formation, reduce postoperative edema, and create a more favorable environment for wound healing. Furthermore, in procedures involving significant tissue resection or manipulation, such as certain abdominal surgeries or joint replacements, drain tubes can be employed to minimize the risk of fluid accumulation and its associated complications. Such an approach enhances patient outcomes by facilitating a more efficient healing process and reducing the potential for postoperative complications.

Surgical drain tubes provide meaningful advantages in managing fluid accumulation post-surgery, but their operation is often associated with several challenges. One of the primary problems is the obstruction of the tube's lumen by blood clots, debris, viscous fluids, and/or other such impediments to fluid flow. These blockages can prevent effective drainage, leading to fluid buildup in the surgical site, which increases the risk of infection, hematoma, or seroma. Such obstructions may require manual manipulation or stripping of the drain to restore patency, a process that can exert excessive force on the tube, risking damage to the tube or trauma to the tissues surrounding the insertion site. Such an exercise can be particularly problematic when carried out by a layperson with no prior experience with surgical drain tubes, which is often the case.

Another significant issue is the maintenance of consistent suction or negative pressure within the system. Surgical drains are often connected to suction devices to facilitate fluid removal, but leaks or blockages can disrupt this negative pressure, rendering the drainage system ineffective. This may necessitate frequent monitoring and adjustment by medical staff, increasing the burden on clinical resources. Additionally, improperly functioning drains can lead to incomplete evacuation of fluids, prolonging recovery time or necessitating further surgical intervention.

Patient discomfort and infection risks are also common concerns. Movement of the drain tube can cause irritation at the insertion site, leading to pain or potential tissue damage. If the tube is not adequately secured or managed, it can become dislodged, further complicating recovery. Infection is another critical risk, as surgical drain tubes provide a potential entry point for pathogens. Proper hygiene, secure placement, and careful management of the drain tube are essential to mitigate these complications, but these measures often require significant diligence and expertise from medical staff and patients alike.

To address the foregoing problems, as well as others, methods and systems such as those described herein provide a surgical drain stripper device that is effective, reliable, and easy to use by medical personnel and non-medical caregivers alike. Such a surgical drain stripper device can be provided by a manufacturer as a standalone device, installable on a surgical drain tube (e.g., after the surgical drain tube has been placed, also allowing for subsequent replacement of the device), as part of a surgical drain tube kit (e.g., where the surgical drain stripper device is pre-installed on the surgical drain tube, installed as part of placing the drain tube, or the like, as part of the surgical operation), or pre-installed on the surgical drain tube, for example.

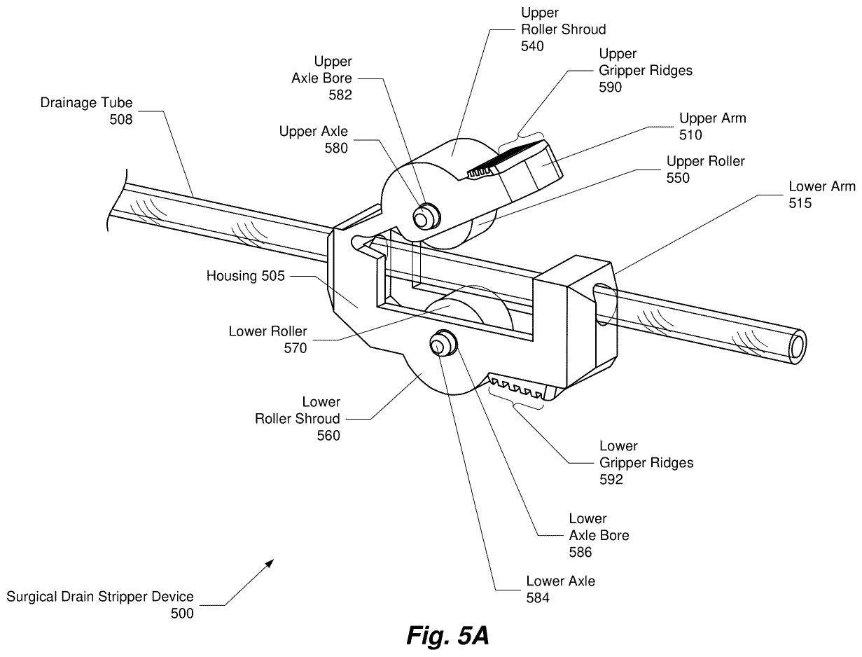

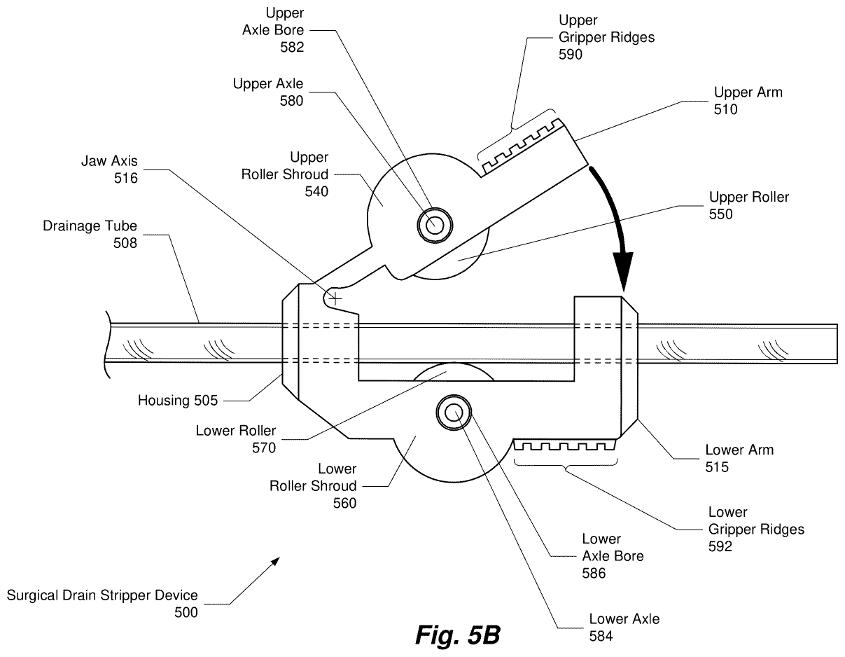

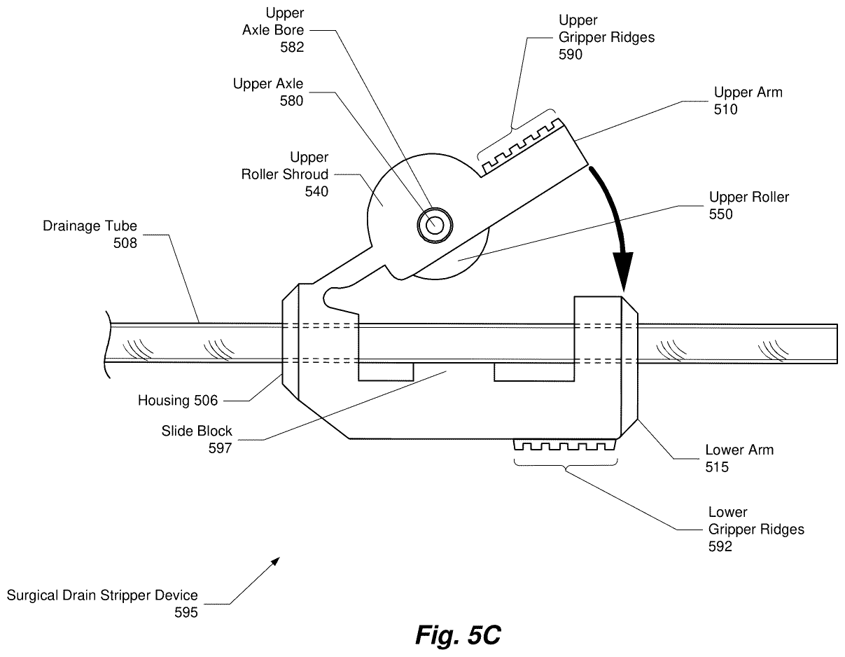

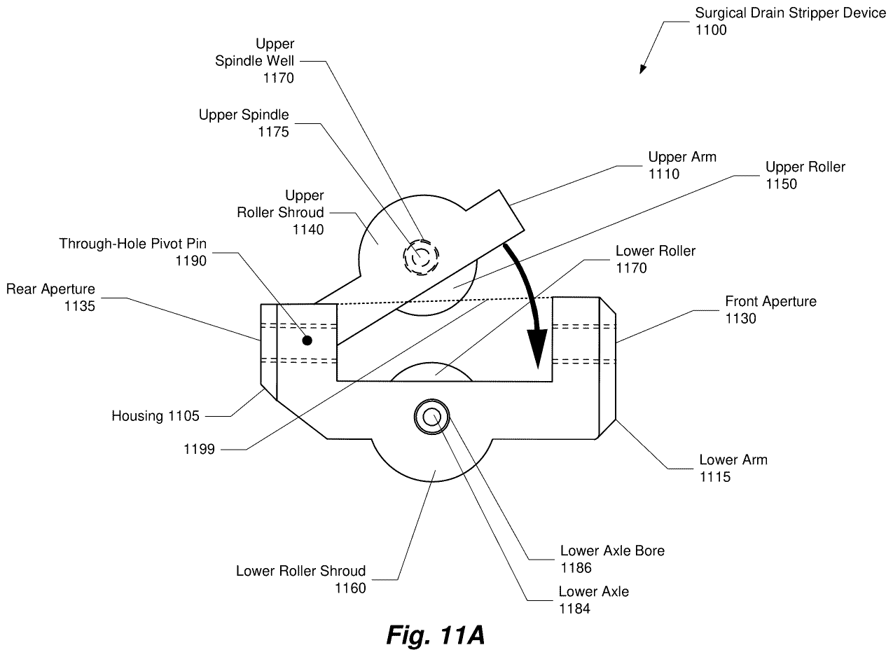

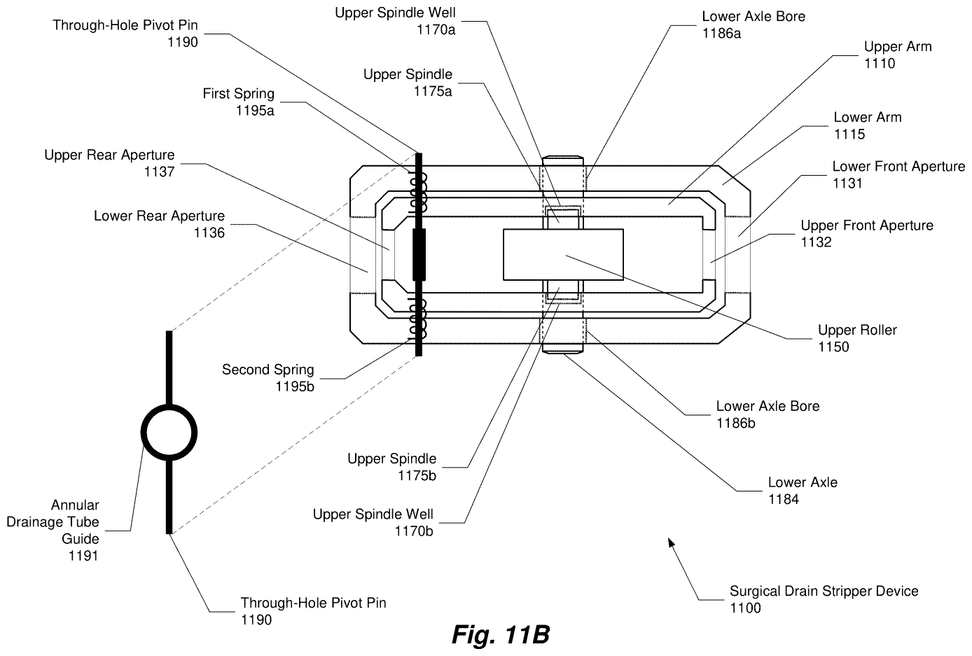

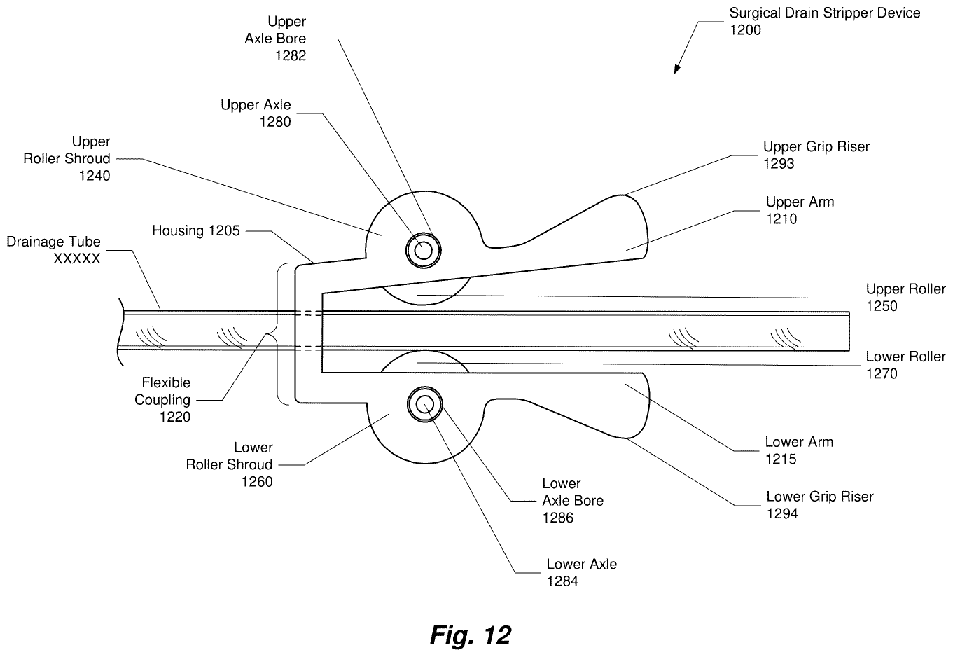

Embodiments such as those described herein provide a surgical drain stripper device that addresses such problems, as well as providing other advantages, including:
Effective Compression—Utilizes rollers and arm mechanisms to apply consistent and controlled pressure to a drainage tube, effectively clearing clots, debris, and sluggish fluids. Supports full lumen compression, and in so deforming the drain tube, clear obstructions/debris without damaging the tube. By compressing the drain tube and, upon release of such compression, allow elastic recovery of the drain tube, additional suction can also be provided by a device according to the present disclosure, as the tube returns to its original shape.
User-Friendly Features—Includes apertures and guides (e.g., annular drainage tube guides) for alignment and unobstructed passage of the drainage tube. Integration of spring-assisted hinge mechanisms (as will be discussed in connection with FIG. 11B) ensures controlled movement and consistent opening positions for ease of use.
Ergonomic Design—Features such as grip-enhancing elements (e.g., the gripper ridges that will be discussed in connection with FIGS. 5A, 5B) or grip risers (as will be discussed in connection with FIG. 12) for improved user handling and control. Optimized for ease of use in clinical scenarios, reducing operational strain and enhancing safety.
Adaptable Rotational Mechanisms—Incorporates various roller support systems (e.g., pin-and-well, spindle, and axle designs) tailored for specific functional and structural requirements. Allows integration of distinct mechanisms for upper and lower rollers (as will be discussed in connection with FIGS. 11A and 11B).
Precision Manufacturing—Supports fabrication via 3D printing with advanced features like frangible tabs for alignment and stability during manufacturing and transport. Ensures tight tolerances and smooth operation with deburring and polishing processes.
Material Versatility—Accommodates biocompatible and sterilizable thermoplastics, enhancing safety and reusability. Lightweight construction provides ease of handling without sacrificing durability.
Customizable and Scalable—Modular design allows for customization of roller sizes, arm lengths, and compression features to suit various tube dimensions and medical needs. Suitable for both prototyping and mass production, ensuring flexibility in clinical applications.
Durability and Maintenance—Durable construction materials and components resist wear, impact, and corrosion, ensuring longevity. Facilitates maintenance with replaceable rollers and deburred, polished features for smooth operation.
Clinical Efficiency—Enhances patient care by ensuring reliable and quick clearance of obstructions in surgical drainage tubes. Minimizes risks associated with manual manipulation of the tube, such as tube damage or incomplete clearing.

As will be appreciated then, a stripper device such as that described herein facilitates the efficient, effective, and reliable clearing of drain tubes. Further, such a device is easy to use, and reduces the risk of damage to the drain tube and tissues surrounding the insertion site, which can be of particular importance when operated by non-medical personnel such as caregivers. Further still, such a device can be readily manufactured, at low cost and in large quantities, due to its design.

Manufacturing of such stripper devices can employ additive manufacturing techniques such as three-dimensional (3D) printing techniques to good advantage. As will be appreciated, 3D printing is a process that fabricates three-dimensional objects layer by layer, following a digital design model. The process begins with a digital 3D model created using computer-aided design (CAD) software or derived from a 3D scan. This model is then converted into a format interpretable by a 3D printer, such as an STL (stereolithography) file. The file undergoes slicing, where specialized software divides the model into horizontal layers and generates instructions (G-code) that guide the printer during fabrication.

The core principle of 3D printing involves depositing or solidifying material in successive layers, starting from the bottom and building upwards. There are various methods of 3D printing, each suited to specific materials and applications. For instance, fused deposition modeling (FDM) extrudes melted thermoplastic through a heated nozzle, selectively depositing it on a build platform layer by layer. Stereolithography (SLA) uses a laser or UV light to cure liquid resin into solid layers. Selective laser sintering (SLS) employs a laser to fuse powdered material, while direct metal laser sintering (DMLS) does the same with metal powders. Each layer adheres to the one below, forming a monolithic object.

The process of 3D printing is characterized by its precision, flexibility, and efficiency, enabling the creation of complex geometries, internal structures, and intricate details that traditional manufacturing methods might not achieve. It reduces material waste compared to subtractive methods like machining, as it uses only the necessary material to build the object. Of particular note is the application of this technology healthcare (e.g., surgical tools), and more specifically, to its use in producing a surgical drain tube stripper and/or components thereof.

In view of the following points and those discussed subsequently herein, it will be appreciated that the present disclosure describes stripper devices that provide for the effective and reliable clearing of drain tubes, as well as methods for manufacturing such apparatus and an apparatus manufactured using such methods.

### Examples of Surgical Drain Stripper Devices

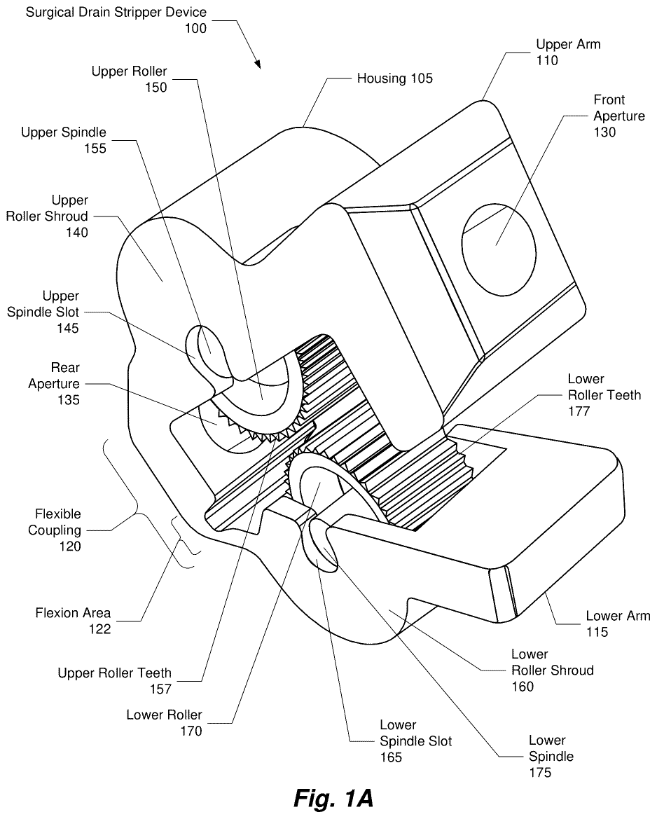

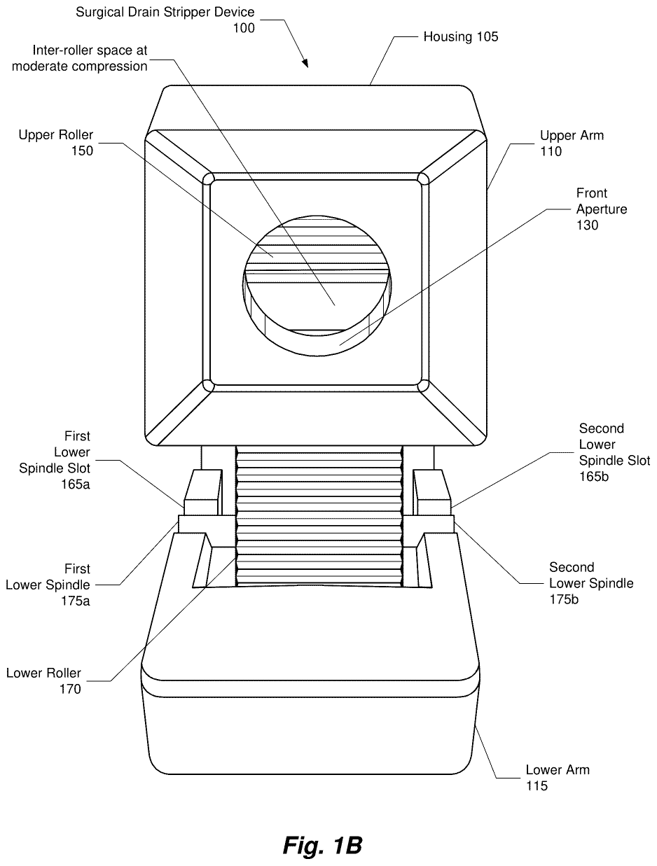

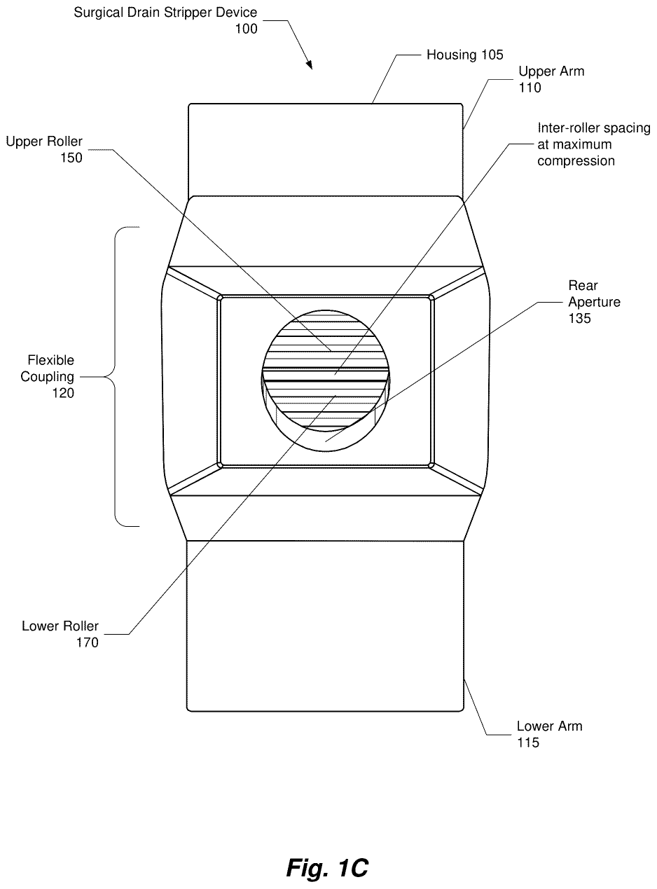

FIG. 1A is a simplified diagram illustrating a perspective view of a surgical drain stripper device having a flexion area at an lower portion of a flexible coupling, according to embodiments of methods and systems such as those disclosed herein. FIG. 1A thus depicts a surgical drain stripper device 100 suitable for stripping (cleaning) a drain tube such as a surgical drain tube, according to such embodiments. Surgical drain stripper device 100 includes a housing 105, which is formed of an upper arm 110 and a lower arm 115. Upper arm 110 and lower arm 115 are mechanically coupled to one another by a mechanical coupling such as a flexible coupling (e.g., depicted as a flexible coupling 120 in FIG. 1A). Flexible coupling 120 features flexion area 122, enabling controlled flexion between a first end of each of upper arm 110 and lower arm 115 (at their respective ends coupled thereto), and the other ends thereof being able to be brought together thereby. Flexion area 122 allows upper arm 110 and lower arm 115 to rotate about a jaw axis at a “rear” “end” of housing 105 (not shown; the terms “rear” and “end” being used as simply terms to allow discussion of the various portions of surgical drain stripper device 100 relative to one another, as described more fully subsequently). The location of such jaw axis varies in position, as a result of the position and dimensions of flexion area 122 within flexible coupling 120 (and so, housing 105), the materials used to produce surgical drain stripper device 100, the dimensions of surgical drain stripper device 100, and other such factors.

Upper arm 110 includes a front aperture 130 and a rear aperture 135, providing openings through which a surgical drain tube may be passed. It will be appreciated that, given that the surgical drain tube can be passed through front aperture 130 and a rear aperture 135 as part of assembling a surgical drain tube apparatus (e.g., an apparatus that can include the surgical drain tube, a suction bulb, and surgical drain stripper device 100), either as an assembled apparatus, as a kit (e.g., supplied in a sterilized container) and assembled as part of the surgical procedure placing the surgical drain tube in question, or in another form. In embodiments manufactured by way of 3D printing, 3D printed embodiments can not only be implemented using 3D printing materials that can be sterilized, but the entire 3D printing process can be incorporated into a process of manufacturing the surgical drain tube apparatus (e.g., as a sterilized kit) in the sterilized environment of that process. As will also be appreciated in light of the present disclosure, surgical drain stripper device 100 can create additional suction beyond that created by the suction bulb.

Upper arm 110 further incorporates an upper roller shroud 140. In the embodiment shown in FIG. 1A, upper roller shroud 140 has an upper spindle slot 145 created therein (e.g., as by deposition (e.g., as by 3D printing techniques), forming/molding (e.g., as by injection molding), machining (e.g., as by computational numeric control (CNC) machining), or other such operations), which allows for the mounting of a roller. To this end, upper roller shroud 140 houses an upper roller 150, which includes a spindle on each side thereof (an example of which is depicted in FIG. 1A as an upper spindle 155). Upper roller 150 includes spindles on each side, one of which is depicted as an upper spindle 155 in FIG. 1A. Upper roller 150 features a contact surface that, in the embodiment depicted in FIG. 1A, includes upper roller teeth 157. Upper roller teeth 157 provide traction against a surgical drain tube when compressed between rollers and serve to move, dislodge, or break up clots, sluggish fluid flows, and other such debris within the drain tube. The design of upper roller teeth 157, including their size, spacing, and depth, is optimized for compatibility with surgical drain tube dimensions and for providing effective clearing of debris from the given drain tube.

Lower arm 115 similarly includes a lower roller shroud 160, which, in turn, includes a lower spindle slot 165 for mounting lower roller 170. Lower roller 170, like upper roller 150, is supported by spindles, with one example depicted as a lower spindle 175. Int eh embodiment depicted in FIG. 1A, lower roller 170 also features a contact surface with teeth (depicted in FIG. 1A as lower roller teeth 177), which interact with upper roller teeth 157 to compress the lumen of a surgical drain tube positioned therebetween, and so provide the aforementioned clearing functionality. In certain embodiments, lower roller teeth 177 can be designed to complement upper roller teeth 157, ensuring consistent traction and effective debris clearance while minimizing the risk of tube damage. The spacing and size of teeth such as upper roller teeth 157 and lower roller teeth can be 177 can be selected based on the compliance of the drain tube's material and size, the amount of compression deemed appropriate (e.g., whether the drain tube should bend over the teeth under full compression, whether the drain tube's lumen should fully close, and other such considerations), the depth of the bend(s) caused by the teeth and the amount by which they are to mesh (e.g., contact ratio), and other such factors, considering the feature size producible by the chosen fabrication method (e.g., the pitch of the 3D printing process), the appropriate level of traction on the drain tube, the ability of the selected dimensions to break up debris (e.g., clots) in the drain tube, and other such factors. In one embodiment of surgical drain stripper device 100, for example, dimensions that can be used for the teeth are a height (or conversely, depth) of ~0.05 mm to ~0.2 mm, and a spacing of ~0.05 mm to ~0.3 mm. Such dimensions can also be in terms of the wall thickness, material (e.g., the material's hardness or compliance), and/or gauge of the tubing (e.g., for a given gauge/material, a height and spacing of 100% of the wall thickness). It is to be appreciated that upper roller 150 and lower roller 170 can be implemented as prefabricated components (e.g., rollers manufactured to the desired dimensions by way of the manufacturing processes noted elsewhere herein, and can use materials such as nylon, polycarbonate, polypropylene, stainless steel, aluminum, or other appropriate materials), prior to or after the 3D printing of upper arm 110 and lower arm 115.

The rollers, upper roller 150 and lower roller 170, are rotatably supported by a general mechanism referred to herein as a shaft. Such a shaft broadly encompasses several configurations, including axles, spindles, and pins, each of which provides specific features depending on the application. In certain embodiments, such an axle may be a rod or comparable component separate from the roller, passing through a central bore in the roller to enable rotation. In other embodiments, such a spindle, as depicted in FIG. 1A (e.g., upper spindle 155 and lower spindle 175), may be integrated into the roller itself, with ends that fit into complementary slots or wells in the respective roller shrouds. In yet other embodiments, such a pin may extend from the housing and fit into corresponding recesses in the rollers, providing another mechanism for rotational support.

The smoothness of action in the rotational mechanism, whether implemented as an axle, spindle, or pin, helps to provide the improved performance offered by surgical drain stripper device 100. Smooth rotational action enhances the control of compression applied to the surgical drain tube, ensuring consistent force along the tube's length. This smoothness translates into a more seamless and smooth operation when clearing clots, debris, and/or sluggish fluids, for example, from the drainage tube. By minimizing jerks or interruptions in motion, such a rotational mechanism ensures that the device operates effectively while reducing stress on both the drainage tube and the user, not to mention the patient.

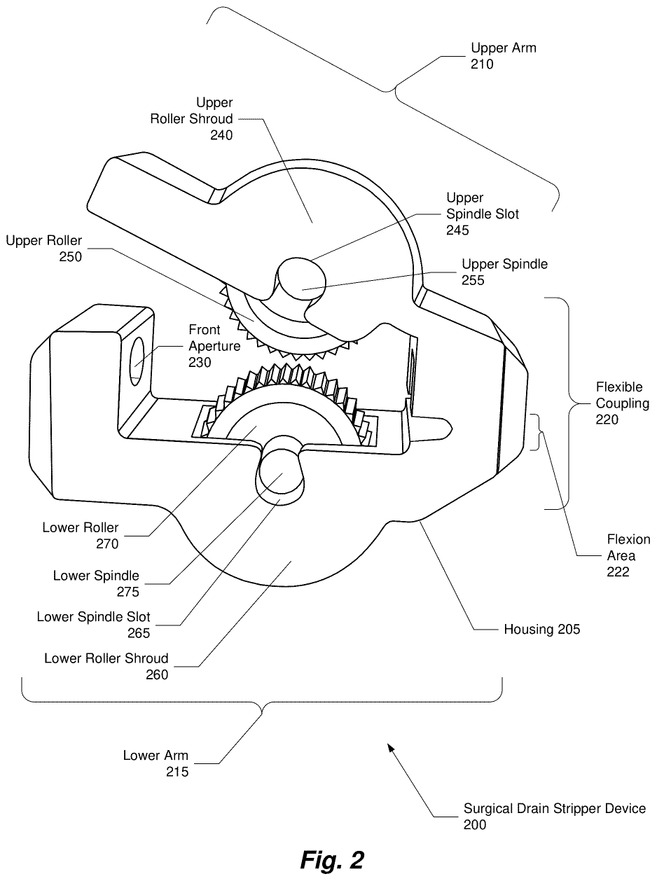

Flexible coupling 120 allows for flexion and alignment of upper arm 110 and lower arm 115, ensuring consistent roller contact and tube compression. Flexion area 122 is configured to withstand repeated flexing motions while maintaining durability, and its dimensions are determined based on the material properties and application requirements of the surgical drain stripper device. Example dimensions of a flexion area such as flexion area 122, as well as materials that can be used to fabricate housing 105 (and so, flexion area 122) are discussed in connection with Table 2, subsequently. Moreover, flexion area 122 can be situated in other locations in the device's housing, as demonstrated by the examples presented in connection with FIGS. 2-7, for example.

Example dimensions of upper are 110 and lower arm 115 with respect to flexible coupling 120 are discussed in connection with Table 2, subsequently, and it is noted that the level of compressive pressure delivered to the surfaces of upper roller 150 and lower roller 170 will depend, in part, on the fulcrum created between flexible coupling 120, and upper roller 150 and lower roller 170.

FIG. 1B is a simplified diagram illustrating an end-on view of one end of the surgical drain stripper device shown in FIG. 1A, according to embodiments of methods and systems such as those disclosed herein. FIG. 1B provides additional detail regarding the spatial and functional relationships between the components of surgical drain stripper device 100 from a perspective looking into the end of surgical drain stripper device 100 referred to herein as the “front” of housing 105 (the opposite end being referred to as the “rear” of housing 105, as described in connection with FIG. 1C, subsequently). Referring to the “ends” of housing 105 in terms of “front” and “rear” is done to simplify this and subsequent discussions, and is not indicative of a direction of travel of surgical drain stripper device 100 along such drain tube, when in use, but is rather referred to in this manner simply for purposes of convenience in describing the drain stripper devices discussed herein.

As before, surgical drain stripper device 100 includes housing 105, which is composed of upper arm 110 and lower arm 115. Upper arm 110 and lower arm 115 are shown positioned to align their respective rollers for effective operation in stripping a drainage tube passed through front aperture 130 (and though rear aperture 135). Also as before, upper arm 110 features front aperture 130, which is visible in FIG. 1B as an opening through which a surgical drain tube may be passed. The alignment of front aperture 130 ensures proper guidance of the drain tube during both insertion of the drain tube into housing 105 and operation of surgical drain stripper device 100.

Upper roller 150 and lower roller 170 are mounted within housing 105. Upper roller 150 is rotatably supported in the assembly of upper arm 110. Similarly, lower roller 170 is mounted within lower arm 115. These rollers are configured with contact surfaces that interact to compress a surgical drain tube positioned in the inter-roller space. FIG. 1B highlights an inter-roller space between upper roller 150 and lower roller 170 at moderate level of compression, where the rollers control pressure applied to the tube with mechanical advantage, as a result of upper arm 110 and lower arm 115 being brought into greater proximity to one another. Housing 105 is preferably designed to provide mechanical advantage sufficient to ensure effective compression of the drain tube, while minimizing the risk of any potential damage thereto (in part, by way of the length of upper arm 110 and lower arm 115, the position of upper roller 150 and lower roller 170 on their respective arms, the flexibility of flexible coupling 120, and the material(s) used, as well as other parameters).

In FIG. 1B, lower roller 170 is supported by rotational mechanisms identified as a first lower spindle 175a and a second lower spindle 175b, in the manner of lower spindle 175 of FIG. 1A. These spindles engage with complementary spindle slots, a first lower spindle slot 165a and a second lower spindle slot 165b. This spindle-and-slot arrangement ensures stable rotational mounting of lower roller 170 within lower arm 115, while allowing lower roller 170 to rotate freely. The precision of this arrangement contributes to the smooth rotational action of lower roller 170, which is critical for applying consistent compression and facilitating the stripping process.

The upper and lower rollers are spaced and aligned to maintain contact sufficient to clear clots, debris, sluggish fluids, and/or other such obstructions from the surgical drain tube passing (inserted) therethrough. FIG. 1B demonstrates the alignment of upper roller 150 and lower roller 170, emphasizing their interaction within the inter-roller space to compress the drain tube effectively.

The end-on view provided in FIG. 1B complements the perspective shown in FIG. 1A by illustrating the alignment and mechanical relationships between rollers, spindles, and apertures. These relationships provide the functionality of surgical drain stripper device 100, ensuring consistent performance during operation, and thereby improving post-operative care.

FIG. 1C is a simplified diagram illustrating an end-on view of the surgical drain stripper device shown in FIG. 1A at an end opposite to that shown in FIG. 1C, according to embodiments of methods and systems such as those disclosed herein. The view shown in FIG. 1C illustrates various details regarding components of surgical drain stripper device 100 as may be visible from the rear of surgical drain stripper device 100, as well as such components' spatial and functional relationships.

That being the case, surgical drain stripper device 100 is depicted in FIG. 1C as including housing 105, which is formed by upper arm 110 and lower arm 115, and the mechanical coupling therebetween provided by flexible coupling 120. Flexible coupling 120 includes a flexion area (e.g., flexion area 122, not visible in FIG. 1C) that allows for controlled relative movement between upper arm 110 and lower arm 115, ensuring alignment and the consistent application of compression to a surgical drain tube positioned between the rollers (e.g., upper roller 150 and lower roller 170, visible in FIG. 1C through rear aperture 135).

Rear aperture 135 and its positioning in the depicted embodiment is more clearly visible in FIG. 1C, and is shown as an opening through the rear of housing 105, potentially adding to the flexibility of flexible coupling 120 (depending on the implementation of housing 105, taking into consideration factors such as the material used in fabricating housing 105, stiffening members added to the exterior/interior of the rear of housing 105 (not shown), the geometry of the angled walls of flexible coupling 120, and other such factors). Rear aperture 135 acts, in the embodiment shown, as a rear-facing guide for positioning and securing a surgical drain tube within surgical drain stripper device 100. The alignment of rear aperture 135 ensures that the surgical drain tube remains properly seated during operation, facilitating effective stripping of the tube.

Upper roller 150 (visible through rear aperture 135) is housed within upper arm 110 and provides a contact surface for interacting with lower roller 170, which is positioned below, in lower arm 115. These rollers are aligned within housing 105 to create an inter-roller spacing therebetween, as depicted in FIG. 1C. Such an inter-roller spacing is shown in FIG. 1C with upper roller 150 and lower roller 170 compressed together at maximum compression for the given embodiment, such maximum compression controlled, in certain embodiments, by upper arm 110 and lower arm 115 coming into contact with one another, one or both of upper arm 110 and/or lower arm 115 coming into contact with stops (as may be fabricated on the same, opposite, or both arms), and/or by other such mechanism. This spacing represents the point of closest proximity between the contact surfaces of upper roller 150 and lower roller 170 in the embodiment shown in FIG. 1C, which allows for application of the necessary force to compress the tube. The spacing is such that clots, debris, sluggish fluids, and the like, can be cleared from the tube while preventing damage to the tube's structure.

Flexible coupling 120 enables the arms to pivot sufficiently to allow for adjustments in the inter-roller spacing depending on the thickness or stiffness of the surgical drain tube, for example. This adaptability ensures uniform compression and enhances the versatility of surgical drain stripper device 100 for various tube sizes and materials.

The view provided in FIG. 1C highlights the alignment of rollers, apertures, and housing components as seen from the rear perspective. The interaction between upper roller 150 and lower roller 170, as mediated by flexible coupling 120, ensures smooth operation and improved drain-clearing performance of surgical drain stripper device 100. The inter-roller spacing and design of the rollers (e.g., teeth) and apertures (e.g., maintaining alignment) contribute to the device's ability to effectively strip and clear surgical drain tubes, maintaining the tube's functionality during post-operative care.

FIG. 2 is a simplified diagram illustrating a perspective view of a surgical drain stripper device having a flexion area at an upper portion of a flexible coupling, according to embodiments of methods and systems such as those disclosed herein. This embodiment, referred to as a surgical drain stripper device 200, demonstrates an alternative arrangement of elements designed to provide functionality and adaptability in the manner of surgical drain stripper device 100, but in a different configuration. In the configuration shown in FIG. 2, it will be noted that the drain tube passes through the “lower” portion of surgical drain stripper device 200, with mechanical coupling in an alternate configuration (both in position and dimensions). It is to be appreciated that, as used throughout the present disclosure, the terms “upper” and “lower” are used simply to distinguish portions of the surgical drain stripper device in question, and so, simplify discussions regarding the features of the given device.

Surgical drain stripper device 200 includes a housing 205, which is composed of an upper arm 210 and a lower arm 215. In the manner of that described earlier, upper arm 210 and lower arm 215 are mechanically coupled to one another by a mechanical coupling (depicted in FIG. 2 as a flexible coupling 220, which includes a flexion area 222). Flexion area 222 is positioned at the “lower” portion of flexible coupling 220 and allows controlled movement between upper arm 210 and lower arm 215. This arrangement facilitates consistency in both alignment and compression of a surgical drain tube positioned between rollers during operation of surgical drain stripper device 200. It is noted here that braces are used for callouts for upper arm 210 and lower arm 215 in order to make evident the general areas of housing 105 that are considered to be, at least potentially, the “arms” of housing 105, there being no need for a strict demarcation between the upper arm, the lower arm, and the mechanical coupling in embodiments such as those described in connection with not only FIG. 2, but others of the figures as well.

As in other embodiments, upper arm 210 incorporates a front aperture 230, visible in FIG. 2 as an opening through which a surgical drain tube may be passed. Front aperture 230 ensures proper guidance and positioning of the tube within surgical drain stripper device 200. Upper arm 210 also includes an upper roller shroud 240, which houses an upper roller 250. Upper roller shroud 240 has an upper spindle slot 245 created therein, which is configured to accommodate an upper spindle 255 of upper roller 250, which rotatably supports upper roller 250 within housing 205 and allows upper roller 250 to rotate with sufficient freedom to allow upper roller 250 to apply adequate pressure to clear a drain tube passing through surgical drain stripper device 200, while avoiding upper roller 250 seizing against upper spindle slot 245.

Also as in other embodiments, lower arm 215 features a lower roller shroud 260, which houses a lower roller 270. Lower roller shroud 260 includes a lower spindle slot 265 fabricated therein, which supports a lower spindle 275 of lower roller 270, allowing lower roller 270 to rotate with sufficient freedom to allow lower roller 270 to apply adequate pressure against upper roller 250 to clear a drain tube passing through surgical drain stripper device 200, while avoiding upper roller 250 seizing against upper spindle slot 245. The alignment of upper roller 250 and lower roller 270 within housing 205 creates an inter-roller space that compresses the surgical drain tube. The contact surfaces of upper roller 250 and lower roller 270 are designed to exert consistent pressure to clear clots, sluggish fluids, or debris without damaging the tube (e.g., as by being toothed with sufficient inter-tooth spacing and tooth depth to perform such clearing, an example of which is depicted in the embodiment shown in FIG. 2).

Flexible coupling 220, with its flexion area 222 positioned at the lower portion of housing 205, and enables dynamic movement between upper arm 210 and lower arm 215. This flexibility allows for control of the compression force applied to the tube, adapting to variations in tube size and material while maintaining smooth operation. As will be appreciated in light of the present disclosure, a smaller flexion area results in reduced compliance of flexible coupling 220, requiring greater pressure to bring the arms together, which is helpful for compressing thicker-walled and/or larger-gauge tubes (those being of lower compliance, and so, needing a sturdier stripper than a higher-compliance tube). Conversely, a device for higher-compliance tubes can use a larger flexion area with less resistance to compression, to provide a more nuanced “feel” when clearing a tube.

Upper arm 210 and lower arm 215, in concert with flexion area 222, provide control over the application of compression force by upper roller 250 and lower roller 270 on a drain tube passing therebetween, and so, effective stripping action on the drain tube. The spindles, including upper spindle 255 and lower spindle 275 (and, while not shown, their counterparts on the other side of upper roller 250 and lower roller 270), interact with their respective spindle slots (e.g., lower spindle slot 265 and upper spindle slot 245) to ensure smooth rotational movement of the rollers. This smoothness translates into seamless compression and clearing action, reducing operational strain on both the user and the surgical drain tube (not to mention the patient). As is discussed subsequently, frangible tabs can be used in certain embodiments to allow upper roller 250 and lower roller 270 to be 3D printed as part of (along with) the 3D printing of housing 205, making production more efficient. Such frangible tabs can be fabricated in the spindle slots, or alternatively, between each roller and its respective roller shroud. Depending on the configuration, such frangible tabs can be fabricated in other locations (e.g., such as between the roller and slide block (in the space in between the arms) for roller/slide block embodiments, such as that shown in FIG. 5C). In yet another alternative, such frangible tabs can be fabricated on the sides of the roller shrouds and spindles, though this can result in the need to 3D print certain such embodiments of the device on its side (such that the frangible portion holds up at least the rollers, but can also include a platform that holds up the housing as well).

FIG. 2 illustrates an arrangement and integration of components in surgical drain stripper device 200 that provides adaptability and efficiency. By positioning flexion area 222 at the lower portion of flexible coupling 220, this embodiment optimizes the balance between rigidity and flexibility, ensuring effective performance across a range of clinical scenarios.

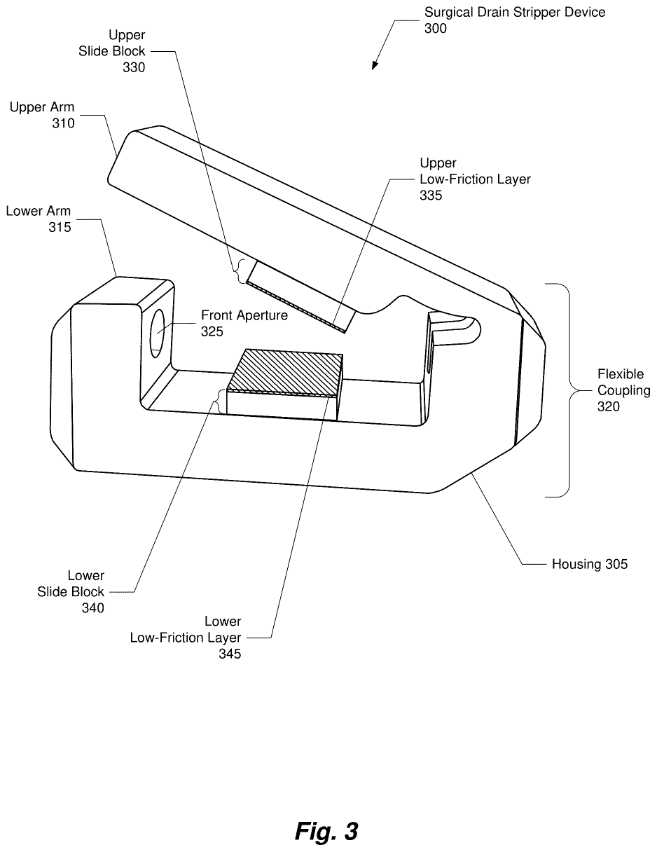

FIG. 3 is a simplified diagram illustrating a perspective view of a surgical drain stripper device employing low-friction materials to form low-friction pads (or low-friction pad against which a roller can compress the drain tube), rather than rollers, according to embodiments of methods and systems such as those disclosed herein. This embodiment, referred to as surgical drain stripper device 300, illustrates a different configuration from those preceding by introducing the use of slide blocks featuring low-friction layers, rather than the rollers illustrated in the preceding figures. That said, it is to be appreciated that slide blocks and rollers such as those shown in the present disclosure can be mixed (e.g., a roller on one arm and a slide block on the other), and be situated on either of the upper or lower arms.

Surgical drain stripper device 300 includes a housing 305, which includes an upper portion (depicted in FIG. 3 as an upper arm 310) and a lower portion (depicted in FIG. 3 as a lower arm 315). These arms are mechanically coupled to one another by a flexible coupling 320. Flexible coupling 320 includes a flexion area located closer to upper arm 310 compared to earlier embodiments (e.g., that shown in FIG. 2). The size of the flexion area (measured by the amount of open space between its jaws) and its positioning (e.g., as where the thickness of upper arm 310 at the flexion area is reduced by such positioning) can be implemented such that the flexion area allows surgical drain stripper device 300 to flex more easily than other configurations depicted in earlier figures. This increased flexibility enhances the device's adaptability for different clinical scenarios and drain tube materials. For example, a flexion area that requires less compressive force to compress upper arm 310 and lower arm 315, as a result of its dimensions and placement, allows for a more nuanced “feel” and feedback when encountering blockages (e.g., clots and debris) and sluggish flow. Such an embodiment also allows for a more incremental application of compressive force to upper arm 310 and lower arm 315 (and so, the pressure applied by the slide blocks of surgical drain stripper device 300), facilitating the clearing of drain tubes made of softer materials and/or having thinner walls, for example.

Upper arm 310 incorporates a front aperture 325, visible in FIG. 3 as an opening through which a surgical drain tube may be passed. Front aperture 325 ensures sufficient guidance and positioning of the tube within surgical drain stripper device 300 to facilitate clearing. Rather than rollers, this embodiment employs an upper slide block 330 and a lower slide block 340, which replace the upper and lower rollers shown in other of the figures. Upper slide block 330 includes an upper low-friction layer 335, while, opposite thereto, lower slide block 340 includes a lower low-friction layer 345. These low-friction layers are fabricated from materials designed to compress a surgical drain tube effectively while preventing the tube from seizing between the slide blocks. This design facilitates smooth passage and effective clearing of clots, debris, sluggish fluids, and the like from the tube.

The interaction between upper slide block 330 and lower slide block 340 creates a controlled compression zone for the surgical drain tube. The low-friction layers, upper low-friction layer 335 and lower low-friction layer 345, ensure that the tube experiences minimal resistance during operation while still applying sufficient pressure to clear obstructions. This approach reduces wear on the tube and enhances the user's control during the stripping process. As will be appreciated in light of the present disclosure, the given slide block can be fabricated separately (either in situ, as part of fabricating the housing, with its low-friction layer applied subsequent to that) or as part of a separate process and subsequently affixed in place (with its low-friction layer applied prior to or after such installation).

The dimensions and position of flexion area of flexible coupling 320, positioned closer to upper arm 310 in the embodiment depicted in FIG. 3 (e.g., in the manner of FIGS. 1A-1C), result in the desired compliance of surgical drain stripper device 300. However, such dimensions and position, along with the dimensions and position of flexible coupling (as well as the rear aperture therethrough), also affect lateral rigidity of housing 305, which, in turn, affects the ability of housing 305 to maintain alignment between upper slide block 330 and lower slide block 340. The flexion area's location and design ensure consistent compression of the tube, while its increased flexibility provides for smoother operation compared to configurations with thicker sections in the related arm, smaller jaw dimensions, or differently positioned flexion areas, which can provide more control in the application of compression (meaningful in situations in which finer or otherwise more delicate drain tubes are used). The trade-off here is that the higher the compliance provided by housing 305 (as well as the other housings described herein), by way of the materials, dimensions, position, and/or other design parameters of any of the arms, slide block(s)/roller(s), flexible coupling, and/or other features of housing 305, the less rigidity housing 305 will have, making the alignment of the slide blocks/rollers less stable than might otherwise be the case, and so, potentially making such high-compliance embodiments less suitable for certain applications (e.g., in the case of more resilient drain tubes). For low-compliance applications (e.g., with drain tubes using stiffer materials and/or thicker tube walls), one alternative is an embodiment in which each arm is a separate piece and the pieces hinged by a through-hole pivot pin. Such an embodiment is described in connection with FIGS. 11A and 11B.

Surgical drain stripper device 300 demonstrates an implementation in which low-friction slide blocks are used in place of rollers. This embodiment can enhanced the device's ability to handle a variety of surgical drain tubes, ensuring effective stripping without certain potential complications associated with roller-based designs (e.g., potentially simpler manufacturing, avoiding the seizing of rollers' shaft mechanisms and the need for smoothing of bores/slots, and so on). That said, slide blocks and rollers such as those shown in the present disclosure can be used in various combinations to good effect, whether in opposition (a roller opposite a slide block or vice versa) and/or a combination of multiple slide blocks and rollers on each arm, in various combinations, as noted.

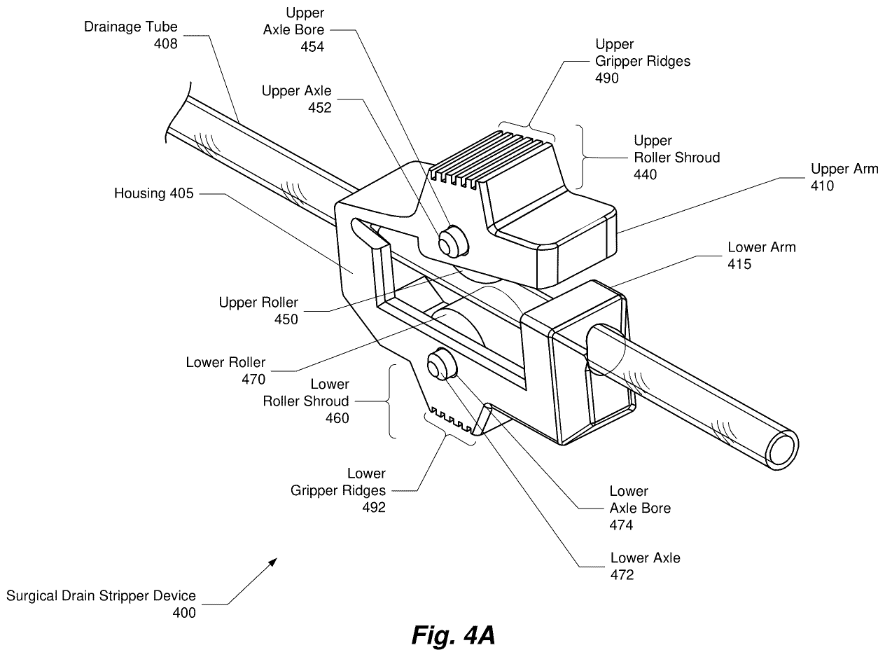

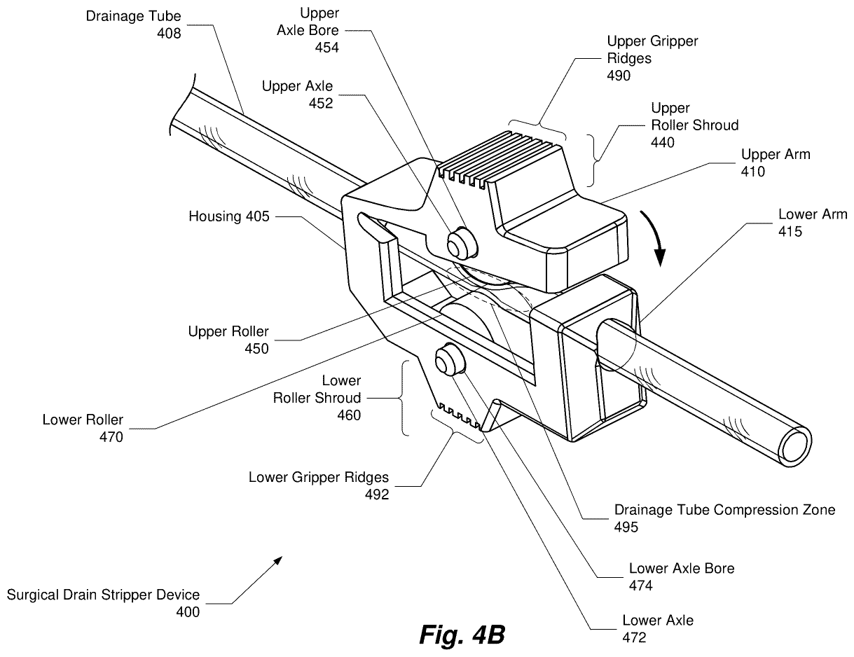

FIG. 4A is a simplified diagram illustrating a perspective view of a surgical drain stripper device that includes a gripper area as part of the roller shrouds, according to embodiments of methods and systems such as those disclosed herein. This embodiment, referred to as a surgical drain stripper device 400, introduces modifications designed to enhance user control when higher compression force is desirable.

Surgical drain stripper device 400 includes a housing 405, which includes an upper arm 410 and a lower arm 415. In the embodiment shown in FIG. 4A, the front and rear apertures are fabricated in lower arm 415, rather than upper arm 410 (the latter as shown in FIGS. 1A-1C, for example). This alternative provides an approach in which the guidance of a drainage tube 408 through surgical drain stripper device 400 does not moved with the arm through which drainage tube 408 passes.

As in earlier figures, upper arm 410 includes an upper roller shroud 440, which houses an upper roller 450. Upper roller 450 is mounted within upper roller shroud 440 using an upper axle 452, which is seated in an upper axle bore 454 (there being another such bore in the other side of upper roller shroud 440 (not shown)). Lower arm 415 features a lower roller shroud 460, which houses a lower roller 470. Lower roller 470 is mounted in lower roller shroud 460 using a lower axle 472 that passes through a lower axle bore 474 (there being another such bore in the other side of lower roller shroud 460 (not shown)). The axles and bores rotationally support upper roller 450 and lower roller 470 in their respective shrouds, enabling smooth rotation of upper roller 450 and lower roller 470 as they interact with one another and drainage tube 408 to compress the lumen of drainage tube 408.

Upper axle 452 and lower axle 472 of surgical drain stripper device 400 can be secured in place using a variety of techniques. For example, upper axle 452 and lower axle 472 can be secured (e.g., by friction, glue, or some other means of attachment) within the bores of upper roller 450 and lower roller 470, respectively. In this embodiment, upper axle 452 and lower axle 472 are free to rotate in upper axle bore 454 (and its counterpart in the other side of upper roller shroud 440) and lower axle bore 474 (and its counterpart in the other side of lower roller shroud 460), respectively. Alternatively, upper axle 452 and lower axle 472 can be secured (e.g., by friction, glue, or some other means of attachment) within upper axle bore 454 (and its counterpart in the other side of upper roller shroud 440) and lower axle bore 474 (and its counterpart in the other side of lower roller shroud 460), respectively, and upper roller 450 and lower roller 470 allowed to freely rotate about upper axle 452 and lower axle 472.

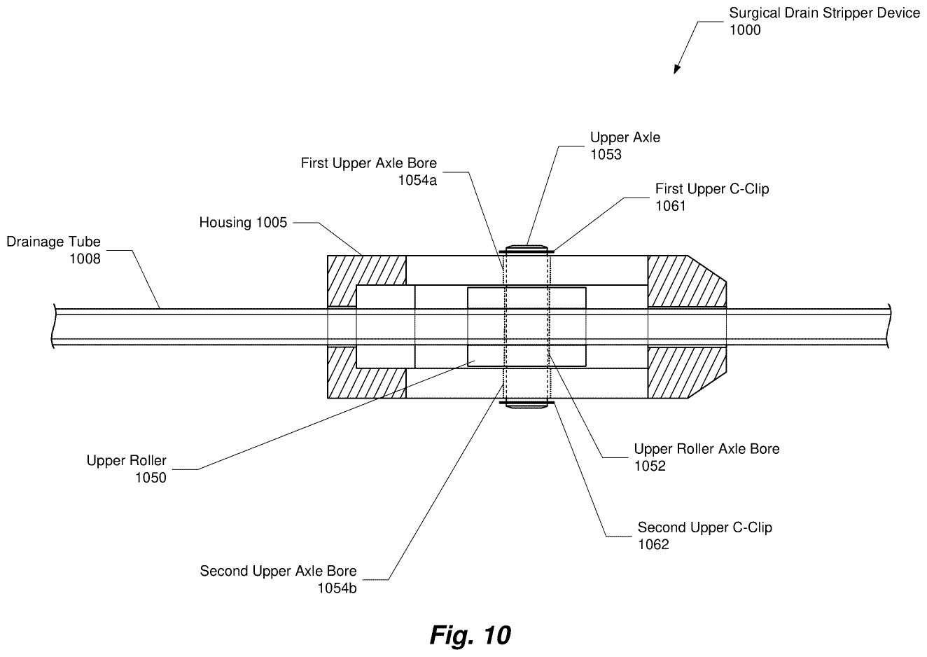

Other alternatives to secure upper axle 452 and lower axle 472 in upper roller 450 and lower roller 470 include fastening mechanisms such as nuts (where the ends of upper axle 452 and lower axle 472 are threaded), machining keyways into upper axle 452 and lower axle 472 (with securing keys inserted therein to lock each axel into its corresponding roller), ring clips (also referred to as C-clips, with grooves created at the ends of upper axle 452 and lower axle 472, in which such ring clips can seat), and/or other fastening mechanisms, in any advantageous combination (none of which are depicted in FIG. 4A). Such mechanisms, if included, can provide extra stability or modularity, allowing for more traditional assembly, maintenance, and/or adaptation to different tube sizes by facilitating the separate fabrication of housing 405, upper roller 450, and lower roller 470 (followed by assembly of surgical drain stripper device 400 by positioning of upper roller 450 and lower roller 470 in housing 405, and the insertion of upper axle 452 and lower axle 472 therethrough, respectively, possibly with the fastening mechanism(s) then being installed). An example of an embodiment in which C-clips are used to secure the axles is described in connection with FIG. 10, subsequently.

As can be seen in FIG. 4A, the rollers used in various embodiments such as those described herein can be smooth (as illustrated in the embodiment in FIGS. 4A and 4B, for example), toothed (as illustrated in the embodiment in FIGS. 1A-1C, 2, and 3, for example), or a combination thereof (e.g., with teeth placed intermittently around the given roller and smooth gaps therebetween). Moreover, such rollers can be used in combination, with one roller being smooth and the other being toothed. These and embodiments employing other such combinations are intended to come within the description provided herein.

In the embodiment shown in FIG. 4A, upper roller shroud 440 includes upper gripper ridges 490, and lower roller shroud 460 includes lower gripper ridges 492. These gripper ridges are strategically positioned to prevent the device from slipping in the user's fingers, particularly when applying greater force to drainage tube 408. Such force may be necessary for tubes with thicker walls, more resilient materials, or larger gauge sizes, for example. Upper gripper ridges 490 and lower gripper ridges 492 provide a textured surface in order to afford additional traction when a user compresses surgical drain stripper device 400, ensuring stable application of compression to drainage tube 408 under varying operational conditions (e.g., when those surfaces would otherwise become unacceptably slippery as a result of bodily (or other fluids) thereon).

Upper roller 450 and lower roller 470 are configured to be brought into proximity with one another as a result of compression of upper arm 410 and lower arm 415 of housing 405, creating a compression zone in drainage tube 408. The rollers' rotational mechanisms, including upper axle 452 and lower axle 472, ensure consistent and smooth operation. This smoothness translates into controlled compression, enhancing the device's effectiveness in clearing clots, debris, sluggish fluids, and/or other such obstructions from drainage tube 408 without damaging drainage tube 408.

The inclusion of gripper ridges in FIG. 4A highlights a key design focus on ergonomics and usability. By enhancing the user's grip and control, surgical drain stripper device 400 is particularly well-suited for challenging scenarios involving resilient or oversized drainage tubes. This embodiment demonstrates an effective combination of functional and user-centric design, making it a versatile and reliable tool for maintaining surgical drain systems.

FIG. 4B is a simplified diagram illustrating a perspective view of the surgical drain stripper device depicted in FIG. 4A, showing the motion involved in compressing a surgical drain tube passing therethrough, according to embodiments of methods and systems such as those disclosed herein. The embodiment of surgical drain stripper device 400 shown in FIG. 4B, as in FIG. 4A, demonstrates an example of the user ergonomics and mechanical actions involved in the operation of surgical drain stripper device 400.

As before, surgical drain stripper device 400 includes housing 405, which includes an upper arm 410 and a lower arm 415. Also as before, upper arm 410 features an upper roller shroud 440, which houses an upper roller 450. Upper roller 450 is rotatably supported by an upper axle 452, positioned within an upper axle bore 454 (with a counterpart bore on the opposite side of upper roller shroud 440, not visible in FIG. 4B). Similarly, lower arm 415 includes a lower roller shroud 460, which houses a lower roller 470. Lower roller 470 is rotatably mounted using a lower axle 472, positioned within a lower axle bore 474 (with a counterpart bore on the opposite side of lower roller shroud 460, not visible in FIG. 4B). Upper axle 452 and lower axle 472 ensure smooth and consistent rotation of upper roller 450 and lower roller 470 as they interact with one another and with drainage tube 408.

In the manner shown in FIG. 4A, upper roller shroud 440 includes upper gripper ridges 490, while lower roller shroud 460 includes lower gripper ridges 492. These gripper ridges are strategically positioned to prevent surgical drain stripper device 400 from slipping during use, particularly when greater compression force is applied. Such force may be necessary when working with tubes made of more resilient materials, tubes with thicker walls, and/or tubes of larger gauge. Upper gripper ridges 490 and lower gripper ridges 492 feature a textured and/or contoured surface to enhance traction, ensuring stable handling even in conditions where fluids may render surfaces slippery.

As in FIG. 4A, upper roller 450 and lower roller 470 are smooth in the embodiment illustrated in FIG. 4B, and are designed to compress the lumen of drainage tube 408 effectively without causing damage. The smooth rollers allow for smoother movement of drainage tube 408 through the compression zone, allowing the device to efficiently, effectively clear clots, debris, and/or sluggish fluids from drainage tube 408.

Also as in FIG. 4A, upper axle 452 and lower axle 472 are secured in their respective rollers and shrouds using techniques such as friction fitting, adhesives, or other fastening mechanisms. Alternatives, such as C-clips, threaded nuts, or machined keyways, can also be employed to enhance the stability and modularity of the device. These fastening methods facilitate easy assembly and maintenance of such embodiments of surgical drain stripper device 400, by way of allowing simple removal of upper axle 452 and lower axle 472, and so, upper roller 450 and lower roller 470. Such an embodiment also provides surgical drain stripper device 400 with a wider range of adaptation to different tube sizes by way of allowing the replacement of the rollers (upper roller 450 and lower roller 470) of one diameter with rollers having a different diameter, in order to allow surgical drain stripper device 400 to accept (and clear) different gauge drain tubes, making surgical drain stripper device 400 a versatile embodiment. It is to be understood that, in light of the present disclosure, the relationship between the diameter of each roller (its roller diameter) and tube gauge, as well as the roller diameter and amount of pressure supplied is a function of the size of the inter-roller space (that distance between the rollers when the device is in its open position), where the larger the space (for a given size tube), the larger the mechanical advantage of the fulcrum formed, and so, the larger the tube size accommodated and the greater the compression force.

As depicted in FIG. 4B, the rollers' rotational mechanisms enable smooth operation, translating compression of upper arm 410 and lower arm 415 into consistent pressure along drainage tube 408. This design ensures that surgical drain stripper device 400 effectively clears blockages without compromising the integrity of the drainage tube. In this embodiment, upper arm 410 and lower arm 415 are configured to move toward one another when compressed by a user, creating a drainage tube compression zone 495 in drainage tube 408. The compression of upper arm 410 (e.g., at upper gripper ridges 490) and lower arm 415 (e.g., at lower gripper ridges 492) towards one another facilitates effective engagement with a drainage tube 408 passing through surgical drain stripper device 400, and creates compression of drainage tube 408 (indicated as drainage tube compression zone 495, as noted). The inclusion of gripper ridges and the design of the compression zone in FIG. 4B demonstrate a commitment to user-friendly and functional design. By combining effective tube compression with enhanced user control, surgical drain stripper device 400 provides a robust solution for maintaining the performance of surgical drain systems in demanding clinical environments.

FIG. 5A is a simplified diagram illustrating a perspective view of another surgical drain stripper device that includes a gripper area toward the end of each arm and using the lower arm as a stop for the upper arm, according to embodiments of methods and systems such as those disclosed herein. This embodiment, referred to as a surgical drain stripper device 500, introduces a design in which the points on the arms at which compressive pressure is applied provides additional control and stability during operation.

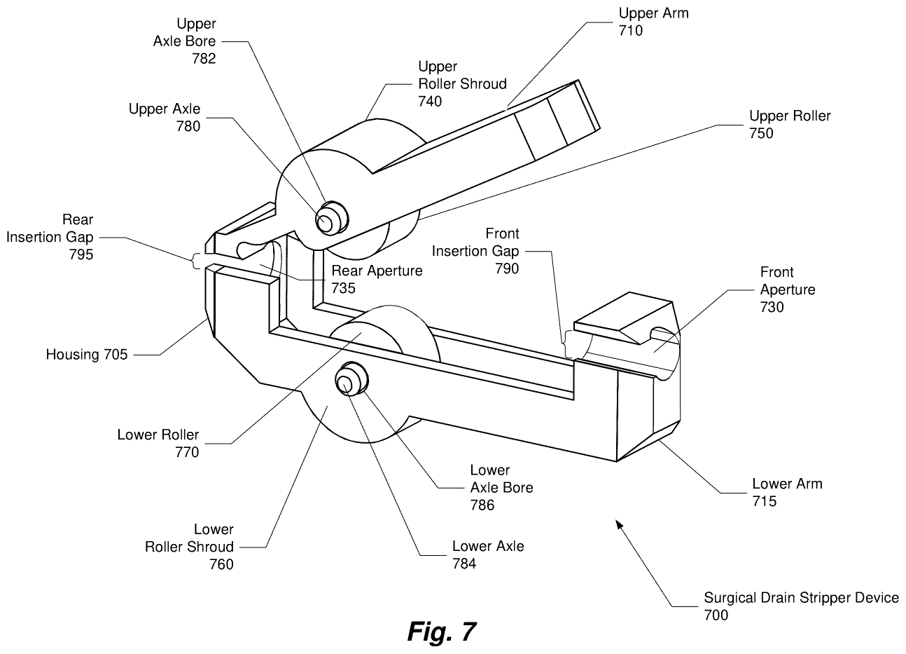

As in earlier figures, surgical drain stripper device 500 includes a housing 505, through which a drainage tube 508 passes. In this regard, it will be appreciated that, in light of the present disclosure, drain tube “kits” can be prepared with a surgical drain stripper device (e.g., such as those described herein), the drain tube, and other associated components, with the surgical drain stripper device already installed on the drain tube. In the alternative, the surgical drain stripper device can be provided as a separate item in such a kit, and the drain tube threaded through the surgical drain stripper device. In fact, a surgical drain stripper device such as that described herein can be offered as a standalone item, for installation on and use with a separately-acquired drain tube and any associated components, by passing the drain tube through the surgical drain stripper device. To this end, as will be discussed in connection with FIG. 7, a surgical drain stripper device can be manufactured as a standalone item that has features designed to allow installation on the drain tube (e.g., after the drain tube and associated components (e.g., a suction bulb) have been assembled and the drain tube placed in a patient).

As before, housing 505 includes an upper arm 510 and a lower arm 515. These arms are designed to move toward each other during operation, enabling compression of drainage tube 508 positioned within the device. Lower arm 515 acts as a physical stop for upper arm 510, ensuring proper alignment and limiting compression to avoid over-application of compressive force to drainage tube 508. Also as before, upper arm 510 includes an upper roller shroud 540, which houses an upper roller 550. Upper roller 550 is mounted within upper roller shroud 540 using an upper axle 580, which is positioned in an upper axle bore 582 (with a corresponding bore on the opposite side of upper roller shroud 540, not visible in FIG. 5A). Lower arm 515 features a lower roller shroud 560, which houses a lower roller 570. Lower roller 570 is mounted using a lower axle 584 positioned in a lower axle bore 586 (with a corresponding bore on the opposite side of lower roller shroud 560, not visible in FIG. 5A). The axles and bores provide rotational support for upper roller 550 and lower roller 570, enabling smooth operation when applying compression to drainage tube 508. These rollers interact within housing 505 to compress the lumen of drainage tube 508 effectively, clearing blockages such as clots, debris, and/or sluggish fluids from drainage tube 508.

Upper roller shroud 540 includes upper gripper ridges 590 and lower roller shroud 560 includes lower gripper ridges 592. These ridges are positioned near the ends of upper arm 510 and lower arm 515, enhancing user control by preventing the device from slipping during operation. The placement of upper gripper ridges 590 and lower gripper ridges 592 allow application of compressive force at the ends of upper arm 510 and lower arm 515. Texturing or shaping of the gripper ridges provides additional traction, ensuring secure handling even when their surfaces are slickened by bodily or other fluids. Thus, upper gripper ridges 590 and lower gripper ridges 592 are particularly useful when higher compression force is required, such as with drainage tubes made of thicker, more resilient materials or larger gauge sizes, as a result of allowing the application of greater compressive force to drain tube 508, while avoiding the risk of the user's fingers slipping off surgical drain stripper device 500. As will also be appreciated, such placement can also be used to provide finer control of the compression force applied by upper roller 550 and lower roller 570 to drainage tube 508.

The rotational mechanisms, upper axle 580 and lower axle 584 in the embodiment illustrated in FIG. 5A, allow for consistent and smooth operation. Upper axle 580 and lower axle 584 can be secured in place using a variety of techniques, such as friction fitting, adhesives, or additional fastening mechanisms like threaded nuts, machined keyways, or C-clips. These mechanisms provide extra stability and modularity, facilitating assembly, maintenance, and adaptation to different tube sizes. The rollers used in surgical drain stripper device 500 are smooth, as depicted in FIG. 5A, and are designed to interact with drainage tube 508 in a controlled manner. The smooth surfaces of the rollers reduce friction, preventing damage to the tube while ensuring effective compression.

By positioning and dimensioning lower arm 515 as a physical stop and including enhanced gripper ridges, surgical drain stripper device 500 highlights a focus on both functional and ergonomic design. These features ensure user control and consistent compression, making this embodiment a versatile and reliable tool for maintaining surgical drain systems in diverse clinical scenarios.

FIG. 5B is a simplified diagram illustrating a side view of the surgical drain stripper device depicted in FIG. 5A, illustrating the passage of a surgical drain tube therethrough, according to embodiments of methods and systems such as those disclosed herein. This embodiment, referred to as surgical drain stripper device 500, demonstrates the functional operation of the device and highlights the interaction of its components during use.

As in earlier figures, surgical drain stripper device 500 includes a housing 505, which is composed of an upper arm 510 and a lower arm 515. These arms are designed to move toward each other during operation, creating controlled compression on a drainage tube 508 positioned within the device. Lower arm 515 serves as a physical stop for upper arm 510, ensuring proper alignment and preventing excessive compression of drainage tube 508. As will be appreciated in light of the present disclosure, it is to be appreciated that the frontispiece of lower arm 515 (that portion rising up from the inner surface of lower arm 515 and through which the front aperture runs) not only serves as a guide for drainage tube 508 and adds structure to housing 505, but also acts as a stop for upper arm 510. The flexion area of housing 505 allows upper arm 510 and lower arm 515 to rotate about a jaw axis 516, which is a virtual line in space, the position of which is affected by factors such as the position and dimensions of the flexion area, the materials used to produce housing 505, the dimensions of surgical drain stripper device 100, and other such factors.

Upper arm 510 features an upper roller shroud 540, which houses an upper roller 550. Upper roller 550 is mounted within upper roller shroud 540 using an upper axle 580. Upper axle 580 is positioned within an upper axle bore 582, which allows for smooth rotation of upper roller 550. A corresponding bore on the opposite side of upper roller shroud 540 (not visible in FIG. 5B) provides additional support.

Similarly, lower arm 515 includes a lower roller shroud 560, which houses a lower roller 570. Lower roller 570 is mounted using a lower axle 584, positioned within a lower axle bore 586. A corresponding bore on the opposite side of lower roller shroud 560 (not visible in FIG. 5B) ensures proper alignment and rotational stability. The axles and bores collectively enable upper roller 550 and lower roller 570 to rotate smoothly, facilitating effective compression of drainage tube 508 as it passes through surgical drain stripper device 500.

Upper roller shroud 540 includes upper gripper ridges 590, and lower roller shroud 560 includes lower gripper ridges 592. These gripper ridges are strategically positioned near the ends of upper arm 510 and lower arm 515 to prevent slipping during operation. This design is particularly beneficial when higher compression force is required, such as with thicker or more resilient drainage tubes. The gripper ridges are textured or shaped to provide additional traction, ensuring secure handling even under conditions where bodily fluids or other substances may render the surfaces slippery.

The rollers used in surgical drain stripper device 500 are smooth, as illustrated in FIG. 5B, to reduce friction and prevent damage to drainage tube 508. The interaction between upper roller 550 and lower roller 570 creates a controlled compression zone, effectively clearing clots, debris, sluggish fluids, and/or other such obstructions from the tube while maintaining its structural integrity.

Upper axle 580 and lower axle 584 can be secured using various techniques, including friction fitting, adhesives, or mechanical fastening mechanisms such as threaded nuts, machined keyways, or C-clips. These mechanisms provide extra stability and modularity, enabling easier assembly, maintenance, and adaptation to tubes of different sizes. The secure attachment of the axles ensures consistent and reliable operation during use.

By incorporating lower arm 515 as a physical stop and enhancing user control with upper gripper ridges 590 and lower gripper ridges 592, surgical drain stripper device 500 exemplifies a focus on both functionality and ergonomics. These features make this embodiment a versatile and effective tool for maintaining surgical drain systems in a variety of clinical settings.

FIG. 5C is a simplified diagram illustrating a side view of the surgical drain stripper device of the design depicted in FIG. 5A, illustrating the passage of a surgical drain tube therethrough, in which a combination of a roller and a slide block is shown, according to embodiments of methods and systems such as those disclosed herein. This embodiment, referred to as a surgical drain stripper device 595, integrates a mixed design using a roller and a slide block for enhanced versatility.

Surgical drain stripper device 595 includes a housing 506, which, as with other embodiments such as those described herein, includes an upper arm 510 and a lower arm 515. These arms are configured to move toward one another during operation, creating controlled compression of drainage tube 508 positioned within the device.

Upper arm 510 includes an upper roller shroud 540, which houses an upper roller 550. Upper roller 550 is mounted within upper roller shroud 540 using an upper axle 580. Upper axle 580 is positioned within an upper axle bore 582, allowing for smooth rotation of upper roller 550. A corresponding bore on the opposite side of upper roller shroud 540 (not visible in FIG. 5C) provides additional support. Upper roller 550 interacts with a slide block 597 in lower arm 515 to form a compression zone for drainage tube 508.

Lower arm 515 includes lower gripper ridges 592 positioned near its distal end. These gripper ridges, along with upper gripper ridges 590 on upper arm 510, enhance user control and prevent slipping during operation, such features are particularly useful when applying higher compression force, such as with thicker or more resilient drainage tubes. The gripper ridges are textured or contoured to provide secure handling, even when their surfaces are affected by bodily or other fluids.

A slide block 597 in lower arm 515 is employed in place of the lower roller seen in previous embodiments. In the embodiment shown in FIG. 5C, at least the inward-facing surface of slide block 597 is constructed from low-friction material, though as noted below, a number of variations are possible, and such variations are intended to come within the present disclosure. Slide block 597 thus interacts with upper roller 550 to compress drainage tube 508. The low-friction surface of slide block 597 reduces resistance and prevents drainage tube 508 from sticking in surgical drain stripper device 595 (a situation referred to herein as seizing) during operation, while ensuring adequate compressive pressure is applied to drainage tube 508 to compress the lumen thereof, sufficient to clear clots, debris, and/or sluggish fluids from drainage tube 508.

It is to be appreciated that, while lower slide block 597 can be fabricated in the manner shown in FIG. 3 (which can be done with the given slide block being fabricated separately (either in situ, as part of fabricating the housing, with its low-friction layer applied subsequent to that) or as part of a separate process and subsequently affixed in place (with its low-friction layer applied prior to or after such installation, although, in the alternative, the slide block can be fabricated entirely from low-friction material), FIG. 5C illustrates an embodiment in which lower slide block 597 is fabricated as part of housing 506. Another aspect of this embodiment is the fabrication of lower slide block 597/housing 506 as a single piece. In such an embodiment, rather than installing/applying a low-friction layer at some point in the fabrication process, lower slide block 597 can be made entirely of low-friction material, and so, fabricated as part of housing 506 without need of a subsequent step of fabrication/application of a low-friction layer. In the alternative, lower slide block 597/housing 506 can be fabricated from low-friction material, making the gripper ridges particularly useful in such embodiments.

It is to be appreciated that such an embodiment simplifies the fabrication of surgical drain stripper device 595 by reducing the number of moving parts. When using 3D printing to fabricate surgical drain stripper device 595, the simpler design results in less complex, faster, and more efficient fabrication (from both a time and materials perspective). Additionally, the features (e.g., teeth) of upper roller 550 (not shown in the embodiment of FIG. 5C) can be made deeper and space farther apart, as they work in opposition to a flat surface (though the surface of slide block 597 can be concave in shape (also not shown), to more closely hew to the shape of upper roller 550). The height of slide block 597 can be designed to provide partial or complete closure of drainage tube 508 (as described elsewhere herein), as can the stop provided by the frontispiece of lower arm 515. The rotational mechanism of upper axle 580 ensures consistent and smooth movement of upper roller 550. Various attachment techniques, including friction fitting, adhesives, or fastening mechanisms like threaded nuts, machined keyways, or C-clips, can be employed to secure upper axle 580. These options provide flexibility in assembly and maintenance while maintaining stability during operation.

Surgical drain stripper device 595 demonstrates a unique configuration by combining a roller and a slide block, optimizing performance for a range of surgical drain tube materials and dimensions. The integration of gripper ridges and the low-friction design of slide block 597 further enhances the device's functionality and user ergonomics, making it a versatile and effective tool for maintaining surgical drainage systems.

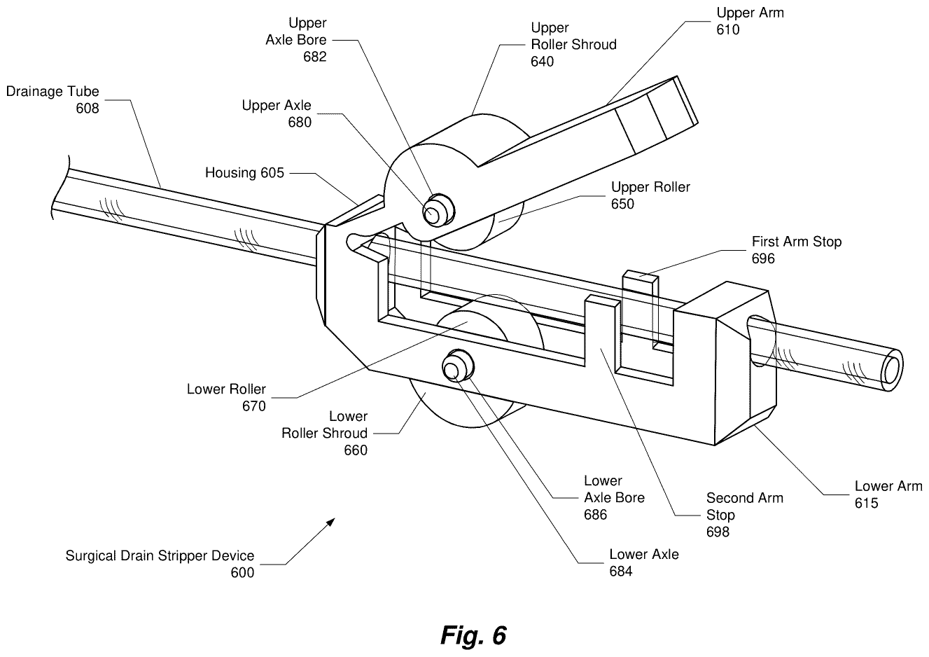

FIG. 6 is a simplified diagram illustrating a perspective view of yet another surgical drain stripper device that includes arm stops on the lower arm to limit motion of the upper arm, according to embodiments of methods and systems such as those disclosed herein. This embodiment, referred to as a surgical drain stripper device 600, incorporates design enhancements that can improve alignment of the drain tube in certain scenarios, and provide greater operational stability by way of arm stops (which, though not illustrated, can be fabricated as part of either arm, one each to the upper and lower arms, and/or of varying dimensions (e.g., in order to add stiffness to maintain structural integrity of surgical drain stripper device 600).

As in earlier figures, surgical drain stripper device 600 includes housing 605, which consists of an upper arm 610 and a lower arm 615. These arms are configured to move toward each other during operation, creating controlled compression for a drainage tube 608 positioned within the device.

Also as before, upper arm 610 includes an upper roller shroud 640, which houses an upper roller 650. Upper roller 650 is mounted within upper roller shroud 640 using an upper axle 680. Upper axle 680 is positioned within an upper axle bore 682, allowing for smooth rotation of upper roller 650. A corresponding bore on the opposite side of upper roller shroud 640 (not visible in FIG. 6) provides additional support. Similarly, lower arm 615 features a lower roller shroud 660, which houses a lower roller 670. Lower roller 670 is mounted within lower roller shroud 660 using a lower axle 684, positioned in a lower axle bore 686. A corresponding bore on the opposite side of lower roller shroud 660 (not visible in FIG. 6) ensures proper alignment and rotational stability. The rotational mechanisms of upper axle 680 and lower axle 684 enable upper roller 650 and lower roller 670 to interact effectively, compressing the lumen of drainage tube 608 as it passes through surgical drain stripper device 600.

Lower arm 615 includes a first arm stop 696 and a second arm stop 698, positioned to limit the motion of upper arm 610. These arm stops serve to ensure proper alignment between upper arm 610 and lower arm 615 during operation, and control the maximum throw of upper arm 610, preventing over-compression of drainage tube 608. By physically restricting the range of motion of upper arm 610, first arm stop 696 and second arm stop 698 enhance operational stability and reduce the risk of damage to drainage tube 608. This feature is particularly beneficial when handling drainage tubes of a particular resilience, thickness, or gauge size.

As just noted, upper arm 610 encounters first arm stop 696 and second arm stop 698, which allows control over the amount of compression of drainage tube 608. By allowing compression of drainage tube 608 only to a known point, for a given set of dimensions of drainage tube 608, surgical drain stripper device 600 provides control over the ultimate amount of closure of the lumen of drainage tube 608, and so, the size of debris cleared from drainage tube 608 (e.g., such control could be anything in the range of slightly closed to completely closed (one side of the lumen of drainage tube 608 seals against the other side thereof). Such control could also be used to prevent the sides of the lumen from sticking to one another (and so interfere with the desired drainage of fluids) by limiting closure to a point sufficient to clear debris of meaningful size, while not allowing those sides to come into full contact with one another. For example, in a situation in which the sides of the lumen might stick to one another (e.g., as a result of the materials used in drainage tube 608, the fluids being drained, and/or a combination of such effects), the dimensions of first arm stop 696 and second arm stop 698 can be selected such that the lumen of the drainage tube in question, being of a certain gauge, only closes to 90% of its original diameter. While this could allow debris smaller than the resulting opening to pass (and so not necessarily be cleared), the closure percentage can be chosen to provide sufficient clearing, without the risk of the drainage tube being closed in upon itself. Control over maximum compression (minimum closure distance) can be particularly important when the user is a layperson (i.e., non-medical personnel), such as a parent or other adult charged with the care of patient in whom drainage tube 608 has been placed, such persons having no experience in this regard (and who are thus more likely to over-compress or under compress surgical drain stripper device 600). Further to this end, though not illustrated, such stops can be spring-loaded (or allowed to move vertically by some comparable method), with graduations thereon indicating too little compression, appropriate compression, and over-compression ranges. In applications where compression in the appropriate compression range provides improved drainage tube clearance, such a feature allows the uninitiated to properly clear the drainage tube while avoiding over-compression thereof.

The rollers used in surgical drain stripper device 600 are smooth, reducing friction and preventing damage to drainage tube 608 while maintaining effective compression. The controlled interaction between upper roller 650 and lower roller 670 creates a compression zone optimized for clearing clots, debris, sluggish fluids, and/or other such obstructions from drainage tube 608. As noted earlier, the types of compression mechanisms used can include roller and/or slide blocks. Such compression mechanisms can be used in place of one another and/or in combination with one another (e.g., either in opposition to one another or in pairs, one pair of a first type at one point along the arms and a pair of another type at another point along the arms).

The axles, upper axle 680 and lower axle 684, can be secured using various attachment techniques, such as friction fitting, adhesives, or fastening mechanisms such as threaded nuts, machined keyways, or C-clips. These options provide flexibility in assembly and maintenance while ensuring stability during operation.

By integrating first arm stop 696 and second arm stop 698, surgical drain stripper device 600 demonstrates a design that provides alignment, control over the amount of compression delivered to drainage tube 608, and operational safety. These features, combined with the effective compression zone and ergonomic design, make surgical drain stripper device 600 a versatile and reliable tool for maintaining surgical drainage systems across diverse clinical scenarios.

FIG. 7 is a simplified diagram illustrating a perspective view of yet another surgical drain stripper device that includes insertion gaps, which allow for the installation of the surgical drain stripper device on a drain tube from the side, according to embodiments of methods and systems such as those disclosed herein. This embodiment, referred to as a surgical drain stripper device 700, introduces a design that permits complete compression of the drainage tube and features side-insertion capability. This allows for a surgical drain stripper device such as surgical drain stripper device 700 to be provided separately from the surgical drain tube apparatus in question, and later installed on its surgical drain tube. This can be helpful not only to allow such devices to be provided separately, but also to be provided as specific to tube sizes and materials, such that a selection may be offered to users. Such features also allow for the replacement of such surgical drain stripper devices (e.g., in the event of such a device breaking or wearing out).

As in earlier figures, surgical drain stripper device 700 includes a housing 705, which consists of an upper arm 710 and a lower arm 715. These arms are configured to move freely toward each other during operation, creating a compression zone for a drainage tube inserted into surgical drain stripper device 700.

Also as before, upper arm 710 includes an upper roller shroud 740, which houses an upper roller 750. Upper roller 750 is mounted within upper roller shroud 740 using an upper axle 780. Upper axle 780 is positioned within an upper axle bore 782, which enables smooth rotation of upper roller 750. A corresponding bore on the opposite side of upper roller shroud 740 (not visible in FIG. 7) provides additional support. Similarly, lower arm 715 includes a lower roller shroud 760, which houses a lower roller 770. Lower roller 770 is mounted using a lower axle 784, which is positioned in a lower axle bore 786. A corresponding bore on the opposite side of lower roller shroud 760 (not visible in FIG. 7) ensures alignment and rotational stability. The rotational mechanisms of upper axle 780 and lower axle 784 enable upper roller 750 and lower roller 770 to interact efficiently, compressing the drainage tube as it passes through surgical drain stripper device 700. Upper axle 780 and lower axle 784 can be secured using various techniques, such as friction fitting, adhesives, or mechanical fastening mechanisms like threaded nuts, machined keyways, or C-clips. These options provide flexibility in assembly and maintenance while maintaining stability during operation. The rollers used in surgical drain stripper device 700 are smooth, ensuring controlled compression while minimizing friction and the risk of damage to the drainage tube. The free movement of the arms and the absence of stops allow the user to more accurately apply incremental force, tailoring compression to the specific requirements of the tube and its contents.

Unlike the previously-described embodiments, surgical drain stripper device 700 does not include arm stops or mechanisms to limit the motion of upper arm 710. In the embodiment shown in FIG. 7, the length of upper arm 710 does not encounter the frontispiece of lower arm 715, and so is able to move past the frontispiece of lower arm 715, and so, completely compress drainage tube 708 (and so, potentially past the point of the inner surfaces of the lumen of drainage tube 708 contacting one another). As a result, upper roller 750 can compress fully against lower roller 770, enabling complete compression of the drainage tube. Such an embodiment can be useful in applications where complete evacuation of drainage tube 708 (including any liquids) is desirable, for example. Complete compression can also be advantageous in certain situations, such as when there is the need to clear particularly resilient obstructions, hardened clots, or thick debris from the drainage tube. This capability provides greater versatility in handling different drainage tube materials and conditions.

The embodiment illustrated in FIG. 7 introduces a front insertion gap 790 and a rear insertion gap 795, located at the respective ends of lower arm 715. These gaps allow for the insertion of the drainage tube into surgical drain stripper device 700 from the side, rather than requiring threading through front aperture 730 and rear aperture 735. This design simplifies the process of inserting or repositioning the tube during operation, enhancing usability in various clinical settings.

It will also be appreciated in light of the present disclosure that use of the appropriate size of surgical drain stripper device can be ensured by appropriately dimensioning front insertion gap 790 and rear insertion gap 795, as well as front aperture 730 and rear aperture 735. For example, given such proper dimensioning, the installation of surgical drain stripper device 700 on drainage tube 708 (e.g., after placement of drainage tube 708 in a patient) can permit its use with certain gauge drainage tubes, but prevent installation on larger gauge drainage tubes (or at least, with significantly increased difficulty, thereby indicating an improperly selected size for the larger gauge drainage tube). This allows surgical drain stripper device 700 to be fabricated with dimensions appropriate to the gauge of drainage tube 708, and so, the amount of compressive force delivered to the compression zone of drainage tube 708. Such control over the installation of surgical drain stripper device 700 by way of the dimensions of front insertion gap 790 and rear insertion gap 795, as well as front aperture 730 and rear aperture 735 (e.g., by fabricating front insertion gap 790 and rear insertion gap 795 at a certain width, and front aperture 730 and rear aperture 735, the gauge of drainage tube that surgical drain stripper device 700 can be installed on can be controlled) thus provides not only improved performance by promoting the use of properly-dimensioned surgical drain stripper devices according to the methods and systems such as those described herein, but also promotes improved patient safety (e.g., as by ensuring the appropriate level of drainage tube clearance). front insertion gap 790 and rear insertion gap 795

It is to be appreciated that front insertion gap 790 and rear insertion gap 795 need not be the same size. rear insertion gap 795 can be smaller than front insertion gap 790, in order to facilitate more securely holding drainage tube 708 in rear aperture 735, which can be important in the stability of the path taken through surgical drain stripper device 700 by drainage tube 708, given that rear aperture 735 is closer to the point of compression (i.e., the compression zone created by upper roller 750 and lower roller 770).

Surgical drain stripper device 700 demonstrates a user-centric design by incorporating insertion gaps and allowing complete compression of the drainage tube. These features, combined with smooth rollers and a free-motion arm design, make surgical drain stripper device 700 an effective tool for addressing a wide range of challenges in maintaining surgical drainage systems.

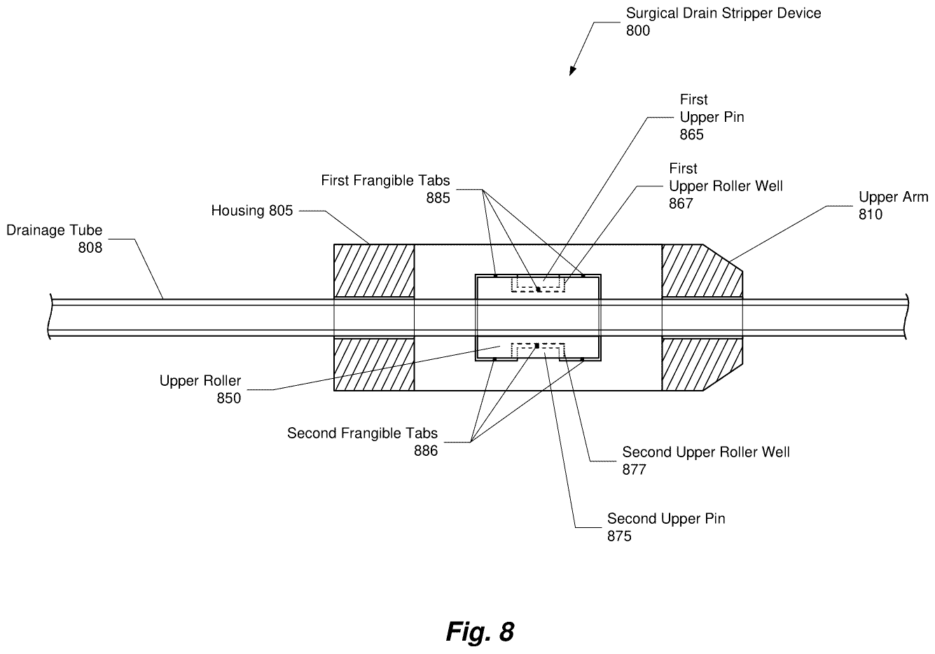

FIG. 8 is a simplified diagram illustrating an overhead cutaway view of a surgical drain stripper device illustrating a roller design, according to embodiments of methods and systems such as those disclosed herein. This embodiment, referred to as a 9, is comparable to that illustrated in FIG. 1A and depicts a configuration designed to provide adaptability and efficient performance through an advanced pin and roller well system, facilitating effective compression and clearance of a drainage tube 808 positioned within surgical drain stripper device 800.

Surgical drain stripper device 800 includes housing 805, which includes an upper arm 810, in the manner illustrated in earlier figures. Housing 805 is designed to allow controlled compression of drainage tube 808 to remove obstructions such as clots, debris, and/or sluggish fluids. Upper arm 810 integrates a roller system that provides reliable and efficient stripping action.

As before, upper arm 810 includes an upper roller 850, which is positioned between a first upper roller well 867 and a second upper roller well 877. To this end, in the embodiment shown in FIG. 8, upper roller 850 is rotatably supported by a first upper pin 865 and a second upper pin 875. These pins extend from housing 805 into their respective roller wells (first upper roller well 867 and second upper roller well 877, respectively) to hold upper roller 850 securely in place while enabling smooth rotational movement of upper roller 850. With regard to the lower arm of housing 805 (not shown), a similar rotational mechanism can be employed, or another of the rotational mechanisms or slide block features comparable to those depicted in other figures presented herein can be used.

First upper pin 865 and second upper pin 875 are, in certain embodiments, cylindrical structures designed to provide rotational support for upper roller 850. These pins are fabricated to ensure that upper roller 850 rotates freely without excessive play or resistance. Each pin is anchored to the internal structure of housing 805 to prevent displacement during operation. The smooth surfaces of first upper pin 865 and second upper pin 875 minimize friction, allowing upper roller 850 to rotate with minimal effort while maintaining consistent pressure on drainage tube 808.

First upper roller well 867 and second upper roller well 877 are recessed features within upper roller 850, and are configured to receive first upper pin 865 and second upper pin 875 of housing 805, respectively. These roller wells are shaped (e.g., in a circular fashion in the embodiment shown) to ensure alignment of upper roller 850 during operation. The depth of first upper roller well 867 and second upper roller well 877 is fabricated to provide sufficient support for the pins, preventing lateral or vertical movement that could disrupt the rotation of upper roller 850, while still allowing upper roller 850 to rotate when applying compression to drainage tube 808. Such rotation is, preferably, sufficiently free to at least permit rotation at a point where, for example, upper arm 850 and the lower arm come into contact with one another, one or both of the arms comes into contact with one or more stops, or the lumen of drainage tube 808 is collapsed in upon itself.

To facilitate such rotation, the walls of the roller wells are preferably fabricated as smooth surfaces, which, in certain embodiments, are coated with a wear-resistant and/or a lower-friction layer (or simply providing such a low-friction surface as a function of the material used, e.g., in the case of housing 805 being fabricated from a low-friction material) to reduce friction and enhance the durability of surgical drain stripper device 800. In certain embodiments, the wells and/or pins can be fabricated in an elliptical shape, in order to produce an eccentric motion as the rollers traverse along the drain tube. It will be noted that the dimensions of the wells and/or pins in such embodiments still need to be such that full and sufficiently free rotation of the rollers is allowed.

Also illustrated in the embodiment of FIG. 8 are first frangible tabs 885 and second frangible tabs 886. First frangible tabs 885 are fabricated between the surface of first upper pin 865 and that of first upper roller well 867 (parallel to the travel of drainage tube 108), while second frangible tabs 886 are fabricated between the surface of second upper pin 875 and that of second upper roller well 877 (also, parallel to the travel of drainage tube 108). First frangible tabs 885 and second frangible tabs 886 facilitate the substantially contemporaneous fabrication of housing 808 and upper roller 850 as part of the same process by providing support for the various portions of upper roller 850 supported thereby during fabrication (e.g., the 3D printing of the portions of upper roller 850 fabricated above the inner surface of housing 808 corresponding to second frangible tabs 886 (and supported thereby), and then stabilized in place by first frangible tabs 885 being fabricated toward the inner surface opposite them, as shown in FIG. 8). First frangible tabs 885 and second frangible tabs 886 therefore can facilitate fabrication of surgical drain stripper device 800 in such manufacturing processes (and, more specifically, housing 805 and upper roller 850 (as well as the lower roller opposite upper roller 850 (not shown as a result of the view illustrated in FIG. 8)). Such a 3D printing process would, in the embodiment shown, fabricate surgical drain stripper device 800 one its side, thus building up portions of housing 805, then second frangible tabs 886 (along with portions of housing 805 at that same depth), then upper roller 850 (along with portions of housing 805 and potentially others of second frangible tabs 886 at that same depth), followed by other portions of housing 805, upper roller 850, and second frangible tabs 886, on a depth-by-depth basis.

Each of the frangible tabs of first frangible tabs 885 and second frangible tabs 886 can then be sheared off one or both of their respective surfaces of upper roller 850 and/or the corresponding surfaces of housing 805 (e.g., at the upper roller well walls) by rotation of upper roller 850 with respect to housing 805, translational movement in opposition to one another, or other such motion as may be sufficient to break the frangible tabs. It is also to be appreciated that the frangible tabs can be anywhere between the roller and housing, so long as the dimensions and location(s) of such frangible tabs are such that only a moderate amount of force is needed to break them off (or there is sufficient clearance to allow for access by a mechanical device to break them, as by cutting). One example is positioning a frangible tabs at the center of first upper pin 865/first upper roller well 867 and second upper pin 875/second upper roller well 877. Such positioning is advantageous because this allows the frangible tabs to be twisted off.

In fabricating the frangible tabs discussed above (e.g., as part of 3D printing surgical drain stripper device 800), and more generally, fabricating surgical drain stripper device 800 by any means (e.g., as by injection molding, computer numerical control (CNC) machining, or other manufacturing processes), the clearances involved in an embodiment such as that shown in FIG. 8, in which first upper pin 865 and second upper pin 875 are circular and rotate within first upper roller well 867 and second upper roller well 877 (also circular), respectively, are preferably maintained within certain limits. For example, the clearance between the roller well and pin on the weight-bearing surface (the surfaces of first upper pin 865/first upper roller well 867 and second upper pin 875/second upper roller well 877 orthogonal to the travel of drainage tube 108) are preferably less than about 2 mm, and most preferably less than about 1 mm. In certain embodiments, a minimum clearance can be ~0.2 mm to ~0.3 mm. Tolerances for such dimensions are within about 0.1 mm to about 0.33 mm, though for smaller clearances, a maximum tolerance of about 20% of the clearance involved is preferable, with about 10% of the clearance involved being most preferable. In such cases, a minimum tolerance of no less than about 10% of the clearance involved is preferable.

On the inner surfaces between first upper pin 865/first upper roller well 867 and second upper pin 875/second upper roller well 877 (parallel to the travel of drainage tube 108, where first frangible tabs 885 and second frangible tabs 886 are located in FIG. 8), the clearance can be less than about 3 mm, for example, but is preferably less than about 2 mm, and most preferably less than about 1 mm. As before, in certain embodiments, a minimum clearance can be 0.3 mm. Tolerances for such dimensions are within about 0.33 mm, though for smaller clearances, a maximum tolerance of about 20% of the clearance involved is preferable, with about 10% of the clearance involved being most preferable. In such cases, a minimum tolerance of no less than about 10% of the clearance involved is preferable. It is to be appreciated that, depending on the dimensions of first frangible tabs 885 and second frangible tabs 886, more clearance between the involved surfaces of first upper pin 865/first upper roller well 867 and second upper pin 875/second upper roller well 877 may be desirable than might otherwise be needed, to allow space for any remaining portion(s) of first frangible tabs 885 and second frangible tabs 886.

The interaction between first upper pin 865, second upper pin 875, and their respective roller wells ensures the stability and reliability of upper roller 850. This configuration allows upper roller 850 to apply consistent pressure to drainage tube 808, effectively compressing and clearing blockages without causing damage to the tube. The secure fit of the pins within the roller wells minimizes vibrations or misalignment, contributing to the overall smoothness of operation.

During operation, upper roller 850 rotates around first upper pin 865 and second upper pin 875 as drainage tube 808 passes through surgical drain stripper device 800. The alignment of the pins within the roller wells ensures that upper roller 850 maintains even contact with the tube, applying sufficient force to clear obstructions while preserving the structural integrity of the tube. Accurate engineering of the pins and roller wells allows for smooth operation, reducing the effort required by the user and enhancing the effectiveness of the device, as well as providing features that facilitate manufacture of surgical drain stripper device 800.

The pin and roller well system in surgical drain stripper device 800 provides reliability and simplicity, which facilitate not only operation but manufacturability. By providing secure yet flexible rotational support for upper roller 850, this configuration ensures consistent performance across a variety of clinical scenarios. The detailed engineering of first upper pin 865, second upper pin 875, first upper roller well 867, and second upper roller well 877 enhances the adaptability and durability of surgical drain stripper device 800, making it an effective tool for maintaining surgical drainage systems.

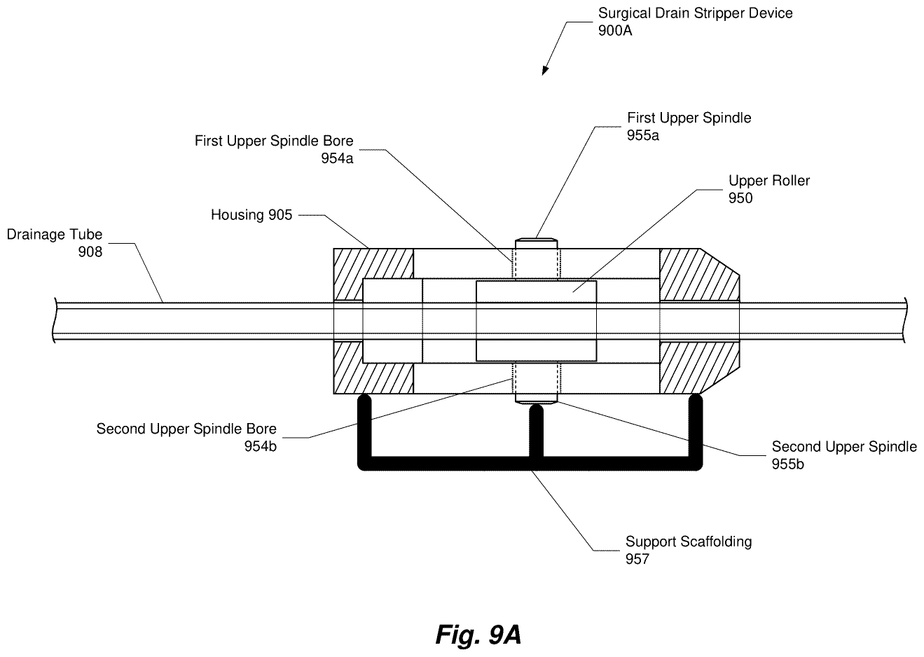

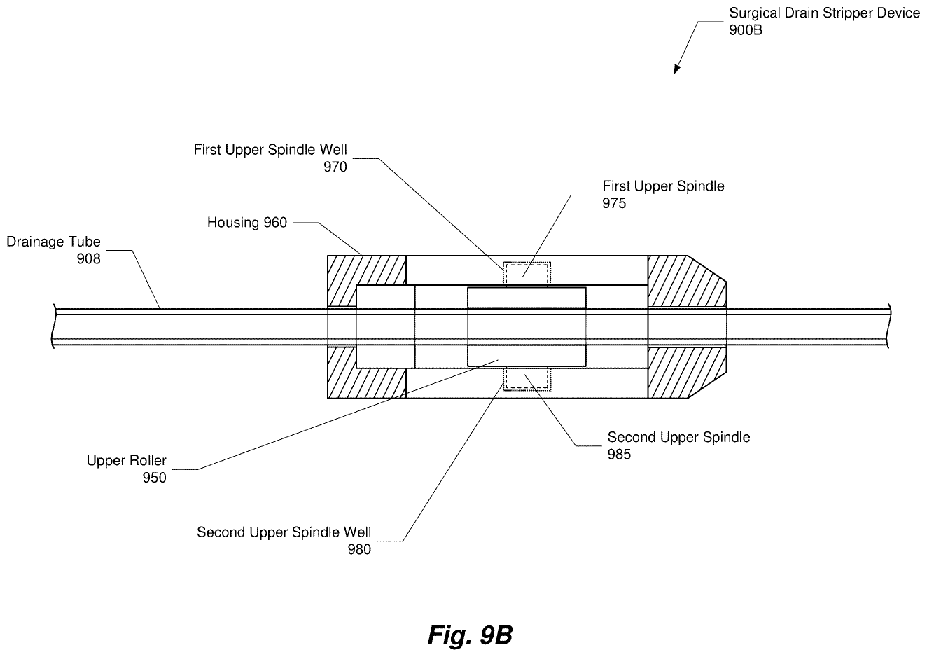

FIG. 9A is a simplified diagram illustrating an overhead cutaway view of a surgical drain stripper device illustrating another roller design, according to embodiments of methods and systems such as those disclosed herein. This embodiment, referred to as a surgical drain stripper device 900A, introduces a configuration where upper roller spindles extend through spindle bores in housing 905, offering an alternative to the pin and roller well system described in FIG. 8, for example.

Surgical drain stripper device 900A includes housing 905, which is the frame for the elements applying compression to a drainage tube 908. Within housing 905, an upper roller 950 is positioned to interact with a lower roller (not shown) to apply compressive force to a drainage tube 908, forming a compression zone that effects removal of obstructions such as clots, debris, sluggish fluids, and/or other such obstructions. Upper roller 950 is supported by a first upper spindle 955a and a second upper spindle 955b, each of which extends through housing 905 via corresponding bores, specifically first upper spindle bore 954a and second upper spindle bore 954b, respectively.

First upper spindle 955a and second upper spindle 955b can be fabricated as an integral part of upper roller 950, fabricated as part of upper roller 950 to ensure accurate and secure operation. In the embodiment shown in FIG. 9A, first upper spindle 955a and second upper spindle 955b are cylindrical and designed to extend outwardly through housing 905, allowing upper roller 950 to rotate freely. This integrated design eliminates the need for separate axles, simplifying the manufacture of surgical drain stripper device 900A. First upper spindle 955a and second upper spindle 955b (and those of the corresponding lower roller, not shown) are fabricated with smooth surfaces to minimize friction and enable consistent rotational movement. Also as before, the clearances and tolerances noted earlier can be implemented to good effect in embodiments such as that shown in FIG. 9A, with regard to the contact surfaces of first upper spindle 955a/first upper spindle bore 954a and second upper spindle 955b/second upper spindle bore 954b.

First upper spindle bore 954a and second upper spindle bore 954b are through-holes in housing 905, and are constructed to accommodate first upper spindle 955a and second upper spindle 955b. These bores are aligned with the axes of the spindles to ensure proper rotational movement of upper roller 950. The dimensions of first upper spindle bore 954a and second upper spindle bore 954b are fabricated such that sufficient clearance for the spindles is provided, while minimizing lateral and vertical play, which could otherwise disrupt operation.

During operation, upper roller 950 rotates around first upper spindle 955a and second upper spindle 955b as drainage tube 908 passes beneath it (and against the roller, slide block, or other feature below drainage tube 908). The spindles' interaction with their corresponding bores ensures that upper roller 950 maintains consistent pressure on drainage tube 908, effectively compressing and clearing blockages without damaging the tube. This design allows for smooth and reliable operation while providing mechanical robustness when using surgical drain stripper device 900A, as well as facilitating manufacture thereof.

When 3D printing techniques are employed, surgical drain stripper device 900A, including housing 905 and upper roller 950, can be fabricated in a single process using support scaffolding 957. Support scaffolding 957 facilitates the simultaneous fabrication of components by providing structural support for elements at various depth levels during the printing process. A depth level, in the context of 3D printing, refers to a specific horizontal plane within the printing volume where material is deposited during a single pass of the printing process. This approach allows for the geometries involved, such as the fabrication of first upper spindle 955a and second upper spindle 955b with upper roller 950, to be achieved without compromising structural integrity, while simplifying the manufacturing process.

To that end, fabricating first upper spindle 955a and second upper spindle 955b as integral elements of upper roller 950, as shown in FIG. 9A, where first upper spindle 955a and second upper spindle 955b are fabricate such that they extend out beyond the outer surfaces of the arms. In such an implementation, 3D printing of surgical drain stripper device 900A employs support scaffolding 957 to hold upper roller 950 (including first upper spindle 955a and second upper spindle 955b) and housing 905 in place during fabrication (e.g., as will be appreciated, given that the housing begins at a “higher” depth than second upper spindle 955b when surgical drain stripper device 900A is 3D printed on its side, some manner of support is needed). It is to be appreciated that, as shown in FIG. 9A, support scaffolding 957 supports these elements frangibly (which can be seen at the narrow points between support scaffolding 957 and upper roller 950/housing 905), such that support scaffolding 957 can be removed with relative ease, once fabrication of surgical drain stripper device 900A is complete, leaving upper roller 950 to rotate freely once support scaffolding 957 is removed. In the alternative, first upper spindle 955a and second upper spindle 955b can be fabricated flush with the respective sides of housing 905 (e.g., the upper shroud of housing 905, in the manner shown in earlier figures), such that 3D printing from the side simply starts with the side of housing 905 and the outer surface of one of first upper spindle 955a or second upper spindle 955b, for example. Such an approach allows 3D printing to begin with either side of surgical drain stripper device 900A.

Further, with regard to fabrication by way of 3D printing, frangible tabs can be fabricated between each spindle and its respective bore, should such support prove desirable to facilitate shipping of surgical drain stripper device 900A, for example (e.g., holding upper roller 950 in place to avoid damage thereto). Such frangible tabs can be broken off and rolled out of the space between the spindle and its bore, as noted earlier. In the alternative, where upper roller 950 is to be installed after fabrication of housing 905, it will be appreciated that housing 905 should be sufficiently flexible to allow insertion of upper roller 950 therein.

The use of support scaffolding 957 during fabrication enables the creation of complex designs, such as first upper spindle bore 954a and second upper spindle bore 954b, with accurate tolerances resulting from the substantial spatial and temporal simultaneity of their fabrication. Once the printing process is complete, support scaffolding 957 can be removed to allow for the full range of motion of upper roller 950. This method of manufacturing simplifies assembly (avoiding the need to install rollers), reduces the material waste of separate fabrication processes (e.g., as between housing 905 and one or two rollers), and ensures consistent performance across surgical drain stripper devices.

It is to be appreciated, however, that support scaffolding such as support scaffolding 957 need not be employed. For example, in certain embodiments, first upper spindle 955a and second upper spindle 955b can be fabricated with their outer ends flush with the sides of housing 905. In this case (and assuming that surgical drain stripper device 900A is fabricated on its side, for example), the first layer of the outer end of second upper spindle 955b and the outer (lower, in FIG. 9A) side of housing 905 can be fabricated, and subsequent layers formed thereon, with each of upper roller 950 and housing 905 supporting subsequent layers thereof, for example. That said, frangible tabs between upper roller 950 and housing 905 can prove helpful in stabilizing these components (e.g., for purposes of shipping, to avoid problems of unwanted component movement during fabrication, and so on).

The integration of first upper spindle 955a and second upper spindle 955b with upper roller 950 and their interaction with first upper spindle bore 954a and second upper spindle bore 954b demonstrate a design focus on precision and reliability. By eliminating separate pins and utilizing a direct spindle-and-bore configuration, surgical drain stripper device 900A enhances operational efficiency and durability, making it a valuable tool for maintaining surgical drainage systems in clinical settings.

FIG. 9B is a simplified diagram illustrating an overhead cutaway view of a surgical drain stripper device illustrating yet another roller design, according to embodiments of methods and systems such as those disclosed herein. This embodiment, referred to as a surgical drain stripper device 900B, introduces a configuration in which an upper roller includes integrates spindles that rest in spindle wells in the housing. Such an embodiment also ensures consistent and efficient compression of a drainage tube positioned within the device.

As in earlier figures, surgical drain stripper device 900B includes housing 960, which forms the structural framework supporting the compressive elements that interact with drainage tube 908. Within housing 960, upper roller 950 is positioned to exert controlled pressure on drainage tube 908, facilitating the clearance of clots, debris, sluggish fluids, and/or other such obstructions. In the manner certain of the earlier-described embodiments, upper roller 950 includes a first upper spindle 975 and a second upper spindle 985, which are fabricated as part of upper roller 950. In contrast to the earlier-described embodiments, however, first upper spindle 975 and second upper spindle 985 of the embodiment illustrated in FIG. 9B rotate in corresponding spindle wells (depicted in FIG. 9B as a first upper spindle well 970 and a second upper spindle well 980, respectively).

As noted, first upper spindle 975 and second upper spindle 985 are integral components of upper roller 950, and are fabricated as part of upper roller 950. Similarly, first upper spindle well 970 and second upper spindle well 980 are fabricated in housing 960. In light of the ability to fabricate these features as part of the same fabricated process (e.g., as by 3D printing), proper alignment and secure rotational functionality can be ensured. As before, first upper spindle 975 and second upper spindle 985 are designed to extend into first upper spindle well 970 and second upper spindle well 980 within housing 960 with clearances and tolerances such as those described earlier with regard to FIG. 8, enabling upper roller 950 to rotate freely during operation. The integration of spindles as part of upper roller 950 simplifies fabrication and provides a mechanism that, as in the other embodiments described herein, ensures consistent performance by reducing free play in the rotation of upper roller 950.

First upper spindle well 970 and second upper spindle well 980 are recessed features within housing 960, configured to securely accommodate first upper spindle 975 and second upper spindle 985. These wells are aligned with the axes of the spindles as a result of being fabricated with upper roller 950 and housing 960, ensuring consistent rotational movement of upper roller 950. The dimensions of first upper spindle well 970 and second upper spindle well 980 provide sufficient clearance for smooth operation while preventing lateral or vertical displacement of upper roller 950 during use, using clearances and tolerances such as those noted earlier. In the alternative, it will be appreciated that housing should be sufficiently flexible to allow insertion of upper roller 950 therein.

During operation, upper roller 950 rotates around first upper spindle 975 and second upper spindle 985 as drainage tube 908 passes beneath it (and between upper roller 950 and the lower roller in opposition thereto (not shown)). The interaction between the spindles and their corresponding wells ensures that upper roller 950 maintains consistent pressure on drainage tube 908, effectively clearing obstructions without compromising the structural integrity of the tube. This spindle-and-well configuration enhances the mechanical stability and durability of surgical drain stripper device 900B.

When 3D printing techniques are utilized to fabricate surgical drain stripper device 900B (including housing 960 and upper roller 950), housing 960 and upper roller 950 can be fabricated in a single process using frangible tabs to temporarily secure the components during manufacturing (e.g., in the manner described earlier). These frangible tabs, located between upper roller 950 and housing 960, provide structural support during the printing process and are designed to break away easily after fabrication, enabling the free rotation of upper roller 950. The use of frangible tabs simplifies the manufacturing process and ensures accurate alignment of the spindles and wells. As with the embodiment shown in FIG. 9A the concept of depth levels in 3D printing, as applied to surgical drain stripper device 900B, refers to the horizontal planes within the printing volume where material is deposited during successive passes of the printing process. By printing surgical drain stripper device 900B layer by layer, with the spindles and wells formed at the appropriate depth levels, the device can be fabricated with precision and consistency.

The integration of first upper spindle 975 and second upper spindle 985 with upper roller 950, along with their interaction with first upper spindle well 970 and second upper spindle well 980, provides ease of manufacture and reliable operation. This design ensures consistent pressure application, smooth rotational movement, and enhanced manufacturability, making surgical drain stripper device 900B a valuable tool for maintaining surgical drainage systems in clinical settings.

FIG. 10 is a simplified diagram illustrating an overhead cutaway view of a surgical drain stripper device illustrating still another roller design, according to embodiments of methods and systems such as those disclosed herein. This embodiment, referred to as a surgical drain stripper device 1000, incorporates an axle-based rotation mechanism to support the upper roller, allowing for consistent compression and efficient clearance of a drainage tube.

As in earlier figures, surgical drain stripper device 1000 includes housing 1005, which provides the structural framework for the mechanisms compression and clearing mechanisms interacting with a drainage tube 1008. Within housing 1005, an upper roller 1050 is positioned to apply controlled pressure to drainage tube 1008 (e.g., in opposition to another roller (supported using the same, similar, or other rotational mechanism) or slide block(s)), facilitating the removal of clots, debris, sluggish fluids, and/or other obstructions from drainage tube 1008. Upper roller 1050 rotates on an upper axle 1053, which extends through corresponding bores in housing 1005 and upper roller 1050, enabling smooth rotational movement.

In the embodiment shown in FIG. 10, upper axle 1053 is separate from upper roller 1050 and housing 1005, allowing upper roller 1050 and housing 1005 to be fabricated separately, which can be advantageous, for example, in a scenario in which upper roller 1050 is fabricated from a low-friction material (e.g., TEFLON, polyethylene, ultra-high molecular weight polyethylene (UHMWPE), acetal (e.g., DELRIN), nylon, and/or other materials with low coefficients of friction (e.g., about 0.02 to about 0.60)) and housing 1005 is fabricated from a non-skid/rigid material (e.g., a thermoplastic polymer such as acrylonitrile butadiene styrene (ABS) plastic, formed with non-skid surfaces such as those noted earlier). Upper axle 1053 passes through a first upper axle bore 1054a and a second upper axle bore 1054b, which are located on opposing sides of housing 1005. These bores are aligned to ensure proper positioning and rotational functionality of upper axle 1053. Additionally, upper axle 1053 passes through an upper roller axle bore 1052, located along the axis of upper roller 1050, providing central rotational support.

To secure upper axle 1053 within housing 1005, a first upper C-clip 1061 and a second upper C-clip 1062 are positioned at each end of upper axle 1053, outside housing 1005. These C-clips prevent axial displacement of upper axle 1053, ensuring stability during operation. The use of C-clips allows for easy assembly and disassembly, facilitating not only manufacture of surgical drain stripper device 1000, but also its maintenance and replacement of its components, as may be needed.

First upper axle bore 1054a, second upper axle bore 1054b, and upper roller axle bore 1052 are fabricated to tolerances such as those noted, facilitating smooth rotation of upper axle 1053 while minimizing lateral and vertical play. The contact surfaces of these bores may include low-friction or wear-resistant coatings, or may be fabricated from inherently low-friction materials, to reduce wear and enhance the longevity of surgical drain stripper device 1000, as can upper axle 1053.

During operation, upper roller 1050 rotates on upper axle 1053 as drainage tube 1008 passes beneath it. The alignment of upper axle 1053 within upper roller axle bore 1052 and housing 1005 ensures consistent application of pressure by upper roller 1050 on drainage tube 1008, effectively clearing blockages while preserving the structural integrity of drainage tube 1008. The axle-based design enhances the mechanical robustness of surgical drain stripper device 1000, providing reliable and efficient performance.

This axle-based rotation mechanism can offer advantages in manufacturing and assembly. The modularity provided by the use of an axle such as upper axle 1053 allows for separate fabrication of upper roller 1050, housing 1005, and upper axle 1053, which can then be assembled using straightforward insertion and clipping techniques. The inclusion of first upper C-clip 1061 and second upper C-clip 1062 simplifies the installation process while ensuring secure and stable operation.

Surgical drain stripper device 1000 provides a reliable and easy-to-use device that is also easy to maintain. The separate fabrication of upper axle 1053 facilitated by the embodiment shown in FIG. 10 also provides consistent performance and durability, making surgical drain stripper device 1000 an effective tool for maintaining surgical drainage systems in various clinical scenarios.

FIG. 11A is a simplified diagram illustrating a side view of a surgical drain stripper device that illustrates an embodiment in which the arms are separate components, according to embodiments of methods and systems such as those disclosed herein. This embodiment, referred to as a surgical drain stripper device 1100, integrates features enabling the arms to hinge about a through-hole pivot pin and demonstrates the use of distinct rotational mechanisms for the upper roller and lower roller, ensuring adaptability and efficient performance across varied clinical scenarios.

As in earlier figures, surgical drain stripper device 1100 includes housing 1105, which forms the structural framework supporting the compressive elements that interact with a drainage tube 1108. Housing 1105 includes an upper arm 1110 and a lower arm 1115. in the embodiment shown in FIG. 11A, lower arm 1115 includes a front aperture 1130 and a rear aperture 1135 that provide guidance of drainage tube 1108, in the manner of shown in earlier figures. Upper arm 1110 includes an upper roller shroud 1140, which houses an upper roller 1150. Upper roller 1150 is rotatably supported by an upper spindle system comprising a upper spindle 1175 (and on the opposite side, a second such spindle (not shown)). These spindles extend from upper roller 1150 into corresponding recessed upper spindle wells (an example of which is upper spindle well 1170 (and on the opposite side, a second such spindle well (not shown)) located within upper roller shroud 1140. The spindle-and-well configuration ensures that upper roller 1150 maintains alignment while rotating smoothly to compress drainage tube 1108. This rotational mechanism provides efficient compression of the tube, clearing obstructions such as clots, debris, and/or sluggish fluids without damaging the tube's structural integrity.

Also as before, lower arm 1115 includes a lower roller shroud 1160, which houses a lower roller 1170. Lower roller 1170 rotates on a lower axle 1184, which passes through corresponding lower axle bores in lower roller 1170 and lower roller shroud 1160 (an example of which is depicted in FIG. 11A as a lower axle bore 1186). Lower axle 1184 is a separate component that provides stable rotational support for lower roller 1170, allowing it to compress drainage tube 1108 effectively during operation, as well as bear the force applied via upper roller 1150. While not shown in FIG. 11A, lower axle 1184 can be secured in place using retention mechanisms such as C-clips at its ends, positioned outside lower roller shroud 1160. This configuration enhances the load-bearing capacity of lower roller 1170 (which can be desirable, given the full compression of drainage tube 1108 provided by surgical drain stripper device 1100), as well as facilitating manufacture, maintenance, and replacement of lower roller 1170, if needed.

In the embodiment illustrated in FIG. 11A (and FIG. 11B, as well), the mechanical coupling employed is by way of a through-hole pivot pin 1190, which is described in further detail in connection with FIG. 11B, subsequently. Here, upper arm 510 and lower arm 515 rotate about a jaw axis that coincides with through-hole pivot pin 1190. In such embodiments, the jaw axis is at the center of through-hole pivot pin 1190, regardless of factors such as those described earlier. As will be appreciated in light of the present disclosure, implementations that employ a through-hole pivot pin such as through-hole pivot pin 1190 allow upper arm 1110 and lower arm 1115 to be fabricated separately and then assembled using through-hole pivot pin 1190, which can be fabricated as a single piece or as a multi-piece component), and creates a hinge point between the two arms (e.g., the arms being coupled to one another by way of through-hole pivot pin 1190 and maintained in an open position by springs such as those depicted in FIG. 11B). Through-hole pivot pin 1190 allows upper arm 1110 and lower arm 1115 to hinge relative to one another during operation. To enhance rigidity, the sides of lower arm 1115 can be designed to be flush across the top, as indicated by dashed line 1199 in FIG. 11A. In such an embodiment, upper arm 1110 hinges into lower arm 1115, which can prevent a user's fingers, clothing, foreign objects, or other impediments when closing surgical drain stripper device 1100.

The rotational mechanisms for the upper and lower rollers differ in this embodiment to allow upper arm 1110 to fold into lower arm 1115, thereby accommodating the requirements of this design. For example, the use of spindles for upper roller 1150 allows upper arm 1110 to pass within lower arm 1115 at the rear portion of housing 1105 (discussed in further detail in connection with FIG. 11B) or entirely therewithin, facilitating full compression of drainage tube 1108, and so, complete closure of the lumen of drainage tube 1108. This arrangement facilitates such complete compression of drainage tube 1108 by ensuring that the rollers contact and fully compress the tube before the arms contact one another, stops, or other structures of surgical drain stripper device 1100.

Through-hole pivot pin 1190 provides the mechanical coupling that enables hinging movement between upper arm 1110 and lower arm 1115. The pivot pin's placement allows the arms to align correctly while maintaining the ability to apply compressive force to drainage tube 1108. The design ensures that upper arm 1110 and lower arm 1115 function in unison during operation, creating a consistent compression zone that effectively clears blockages without excessive strain on the user or the device.

By integrating distinct rotational mechanisms for upper roller 1150 and lower roller 1170, surgical drain stripper device 1100 demonstrates adaptability and versatility. The spindle-and-well system for upper roller 1150 and the axle-and-bore system for lower roller 1170 optimize the performance of each roller based on its placement and functional requirements. These features, along with the modular design and thoughtful engineering of the apertures and pivot mechanism, make surgical drain stripper device 1100 an efficient and reliable tool for maintaining surgical drainage systems in diverse clinical settings.

FIG. 11B is a simplified diagram illustrating an overhead cutaway view of a surgical drain stripper device such as that depicted in FIG. 11A, according to embodiments of methods and systems such as those disclosed herein. In this embodiment, surgical drain stripper device 1100 again employs a design in which the arms and rotational mechanisms for the rollers are distinct, with additional elements facilitating effective compression and structural rigidity.

As in earlier figures, surgical drain stripper device 1100 includes a housing 1105, which supports the compressive elements interacting with a drainage tube 1108. Housing 1105 consists of an upper arm 1110 and a lower arm 1115. Upper arm 1110 and lower arm 1115 each include apertures that guide drainage tube 1108.

Upper arm 1110 includes a lower front aperture 1131 and an upper front aperture 1132, which are depicted as openings through which drainage tube 1108 is guided. Similarly, lower arm 1115 includes a lower rear aperture 1136 and an upper rear aperture 1137, which ensure proper alignment and positioning of drainage tube 1108 as it enters and exits surgical drain stripper device 1100. Upper front aperture 1132 and lower front aperture 1131 provide an entry point for drainage tube 1108, while upper rear aperture 1137 and lower rear aperture 1136 facilitate the tube's exit.

In certain embodiments, upper front aperture 1132 can be designed as a U-shaped slot (or oval aperture), allowing upper arm 1110 to rotate through its arc in contacting and compressing drainage tube 1108. Upper rear aperture 1137 can also be designed as U-shaped slot (or oval aperture), although given the shorter throw of this end of upper arm 1110, the size of upper rear aperture 1137 need not be as vertically large as that of upper front aperture 1132. By contrast, it is to be understood that lower front aperture 1131 and lower rear aperture 1136 will typically be circular and of a diameter to ensure proper passage of drainage tube 1108 through surgical drain stripper device 1100. These apertures' alignment ensures stability in guiding surgical drain stripper device 1100 along drainage tube 1108 (or in guiding drainage tube 1108 through surgical drain stripper device 1100), as well as consistent compression along the compression zone. For example, upper front aperture 1132 and upper rear aperture 1137 can be configured as U-shaped openings, with these apertures open downward. Similarly, lower front aperture 1131 and lower rear aperture 1136 can also (or in the alternative) be configured as U-shaped openings, with these apertures open upward. Such features can simplify the threading of drainage tube 1108 into surgical drain stripper device 1100, as well as the operation of surgical drain stripper device 1100.

The aforementioned apertures, designed as bores (as shown in earlier figures) or slots, for example, can include insertion gaps to allow drainage tube 1108 to be easily inserted or removed. In certain embodiments, insertion gaps can be provided (e.g., such as those shown in FIG. 7) to allow insertion/extraction of drainage tube 1108 from the side of surgical drain stripper device 1100, such that their insert gaps match on either or both ends. This constrains drainage tube 1108 to being inserted/extracted at a certain point of compression of upper arm 1110 and lower arm 1115 (or in the position where upper arm 1110 and lower arm 1115 are the farthest apart). Further, the alignment of such insertion gaps in upper front aperture 1132 and lower front aperture 1131, and those in upper rear aperture 1137 and lower rear aperture 1136, can be designed to align at differing points in the arc through which upper arm 1110 and lower arm 1115 rotate. In such an embodiment, drainage tube 1108 is inserted into (or removed from) surgical drain stripper device 1100 at one point of rotation for upper rear aperture 1137 and lower rear aperture 1136, and another point of rotation for upper front aperture 1132 and lower front aperture 1131, such that drainage tube 1108 is retained in surgical drain stripper device 1100 because the insertion gaps in upper arm 1110 and lower arm 1115 are not all aligned at one point.

Upper arm 1110 features an upper roller 1150, which is rotatably supported by an upper spindle system. This system includes a first upper spindle 1175a and a second upper spindle 1175b, both of which extend outward from upper roller 1150 into corresponding recessed upper spindle wells 1170a and 1170b, respectively. The spindle-and-well mechanism secures upper roller 1150 within upper arm 1110, allowing for smooth rotational movement during operation. This configuration facilitates efficient compression of drainage tube 1108, effectively clearing blockages such as clots, debris, and sluggish fluids.

Lower arm 1115 houses a lower roller 1170, which rotates on a lower axle 1184. Lower axle 1184 passes through corresponding lower axle bores 1186a and 1186b in both lower roller 1170 and lower roller shroud 1160 (not shown explicitly in FIG. 11B). Lower axle 1184 is a separate component that provides robust rotational support for lower roller 1170. While not shown, lower axle 1184 can be retained using mechanisms such as C-clips at its ends, ensuring stability and ease of maintenance. The axle-and-bore system enhances the load-bearing capacity of lower roller 1170, which is beneficial for achieving complete compression of drainage tube 1108.

Upper arm 1110 is connected to lower arm 1115 via a through-hole pivot pin 1190, enabling the arms to hinge relative to each other during operation. Through-hole pivot pin 1190 is a structural component that provides alignment and supports the hinging movement while maintaining the relative positions of the arms. Through-hole pivot pin 1190 includes an annular drainage tube guide 1191, which is located within housing 1105 and is configured to encircle drainage tube 1108, upon the installation of surgical drain stripper device 1100 on drainage tube 1108. Annular drainage tube guide 1191 provides additional guidance for drainage tube 1108, ensuring proper alignment and smooth passage of drainage tube 1108 through surgical drain stripper device 1100. This feature minimizes the risk of misalignment or damage to drainage tube 1108 during operation, and provides a way for drainage tube 1108 to pass through surgical drain stripper device 1100 without obstruction or excessive deviations, avoiding seizing, damage to drainage tube 1108, or other such problems.

Through-hole pivot pin 1190 is installed during the assembly of surgical drain stripper device 1100, and provides mechanical coupling between upper arm 1110 and lower arm 1115, allowing them to hinge with respect to one another, in order to exert compressive force on drainage tube 1108 at a resulting compression zone, while maintaining alignment. In one embodiment, first spring 1195a and second spring 1195b are mounted on through-hole pivot pin 1190 as part of assembling upper arm 1110 and lower arm 1115 to create housing 1105. These springs provide a resistive force that maintains upper arm 1110 and lower arm 1115 in an open position when not in use. The springs can also be designed to provide a resistive force in opposition to compressive force applied to upper arm 1110 and lower arm 1115, thereby ensuring controlled movement of the arms toward one another during compression. In such scenarios, such control can enhance user control and reduce operational strain on drainage tube 1108, which can be important, for example, when drainage tube 1108 has thinner walls, more delicate material, and/or other characteristics that result in drainage tube 1108 being more fragile than might otherwise be the case. In the alternative (on a per-spring basis, even), first spring 1195a and second spring 1195b can be fabricated as part of upper arm 1110, lower arm 1115, or both. Alternatively, dedicated wells within the arms can accommodate these springs. This modularity simplifies assembly and allows for easier maintenance or replacement of the springs if needed.

By integrating distinct rotational mechanisms for the upper roller and lower roller, surgical drain stripper device 1100 achieves adaptability and efficiency. The spindle-and-well system for upper roller 1150 and the axle-and-bore system for lower roller 1170 are tailored to their respective functional requirements, ensuring optimal performance. The addition of annular drainage tube guide 1191 and the spring-assisted hinging mechanism further enhance the device's reliability and user-friendliness, making surgical drain stripper device 1100 a valuable tool for maintaining surgical drainage systems in diverse clinical scenarios.

FIG. 12 is a simplified diagram illustrating a side view of still another surgical drain stripper device, according to embodiments of methods and systems such as those disclosed herein. This embodiment, referred to as a surgical drain stripper device 1200, incorporates features that enhance user control and precision during operation, specifically through the inclusion of grip risers that improve handling.

As in earlier figures, surgical drain stripper device 1200 includes a housing 1205 through which a drainage tube 1208 passes. Housing 1205 forms the structural framework supporting the compressive elements and guiding drainage tube 1208 during operation. Housing 1205 is composed of an upper arm 1210 and a lower arm 1215. These arms are designed to move toward each other during operation to compress drainage tube 1208, effectively clearing obstructions such as clots, debris, and/or sluggish fluids.

As before, upper arm 1210 includes an upper roller shroud 1240, which houses an upper roller 1250. Upper roller 1250 is rotatably supported within upper roller shroud 1240 by an upper axle 1280. Upper axle 1280 is positioned within an upper axle bore 1282, which is fabricated into upper roller shroud 1240 to provide secure rotational support for upper roller 1250. A corresponding upper axle bore (not visible in FIG. 12) is located on the opposite side of upper roller shroud 1240 (as well as a comparable bore in upper roller 1250), ensuring proper alignment and stability for smooth rotational movement. Similarly, lower arm 1215 includes a lower roller shroud 1260, which houses a lower roller 1270. Lower roller 1270 is mounted on a lower axle 1284, which passes through a lower axle bore 1286 within lower roller shroud 1260. A corresponding lower axle bore (not visible in FIG. 12) is located on the opposite side of lower roller shroud 1260 (as well as a comparable bore in lower roller 1270), providing additional alignment and rotational stability for lower roller 1270. Lower axle 1284, like upper axle 1280, is fabricated as a separate component and may be secured using mechanical retention mechanisms such as friction fitting, adhesives, or fasteners like threaded nuts, machined keyways, or C-clips positioned outside lower roller shroud 1260. These axles and bores collectively enable upper roller 1250 and lower roller 1270 to rotate smoothly, creating a controlled compression zone for drainage tube 1208.

In contrast to embodiments depicted earlier, surgical drain stripper device 1200 includes grip risers (depicted in FIG. 12 as an upper grip riser 1293 and a lower grip riser 1294), which are positioned near the ends of upper arm 1210 and lower arm 1215, respectively. These grip risers extend outwardly and are angled to provide enhanced ergonomic control during operation. Unlike the gripper ridges shown in FIG. 5A and FIG. 5B, grip risers 1293 and 1294 are designed to improve the user's hold on surgical drain stripper device 1200 by offering a larger and more angled surface. In certain embodiments, gripper ridges similar to those in earlier figures may be incorporated onto the outer surfaces of grip risers 1293 and 1294, further enhancing traction and control, especially in environments where bodily fluids or other substances might render those surfaces slippery.

The addition of grip risers 1293 and 1294 also facilitates the application of compressive force at the ends of upper arm 1210 and lower arm 1215, allowing for more accurate control of the compression applied to drainage tube 1208. This design is particularly beneficial when dealing with drainage tubes made of thicker, more resilient materials and/or larger gauge sizes, for example, as such a design ensures consistent and effective compression while minimizing the risk of the user's fingers slipping off surgical drain stripper device 1200.

To enhance the functionality and usability of surgical drain stripper device 1200, a flexible coupling 1220 is integrated between upper arm 1210 and lower arm 1215 within housing 1205. Flexible coupling 1220 facilitates relative movement between the arms, enabling smooth operation while maintaining alignment and stability. The placement of flexible coupling 1220 ensures that upper arm 1210 and lower arm 1215 function in unison, creating a reliable and effective compression zone for clearing blockages in drainage tube 1208. In this embodiment, it is to be noted that no flexion area is fabricated, and so flexing of housing 1205 is spread throughout flexible coupling 1220. With regard to fabrication, it is to be appreciated that frangible tabs are optional in this embodiment (given that axles are shown, but that spindles or pins can be used), as will the fact that frangible tabs/support scaffolding are not needed for axle-mounted rollers (which will typically be separate components (e.g., separate from housing 1205)), as will be appreciated in light of the present disclosure. As noted elsewhere herein, the smoothing of spindle slots can be accomplished by simply rolling rollers with force sufficient to break up remaining portions of any such frangible tabs.

By integrating grip risers 1293 and 1294, along with upper axle 1280 and lower axle 1284 mechanisms, surgical drain stripper device 1200 emphasizes both ergonomic and functional design. These features ensure user control, consistent compression, and enhanced adaptability, making surgical drain stripper device 1200 a versatile and reliable tool for maintaining surgical drainage systems in diverse clinical settings.

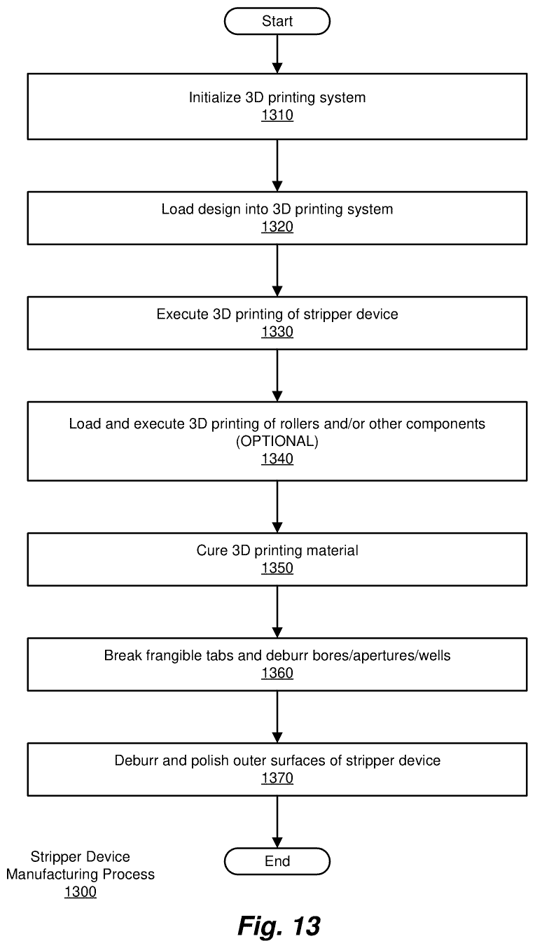

FIG. 13 is a flow diagram illustrating an example of a stripper device manufacturing process, according to embodiments of methods and systems such as those disclosed herein. FIG. 13 thus depicts a stripper device manufacturing process 1300. Stripper device manufacturing process 1300 begins, in the example depicted in FIG. 13, with initializing 3D printing system (1310). Initializing 3D printing system (1310) includes preparing a 3D printing system for the fabrication of surgical drain stripper device 100. This initialization involves tasks such as calibrating the printer's build platform, ensuring filament or resin materials are correctly loaded, and verifying printer settings to match the requirements of the design specifications. This preparation ensures precision in the subsequent operations.

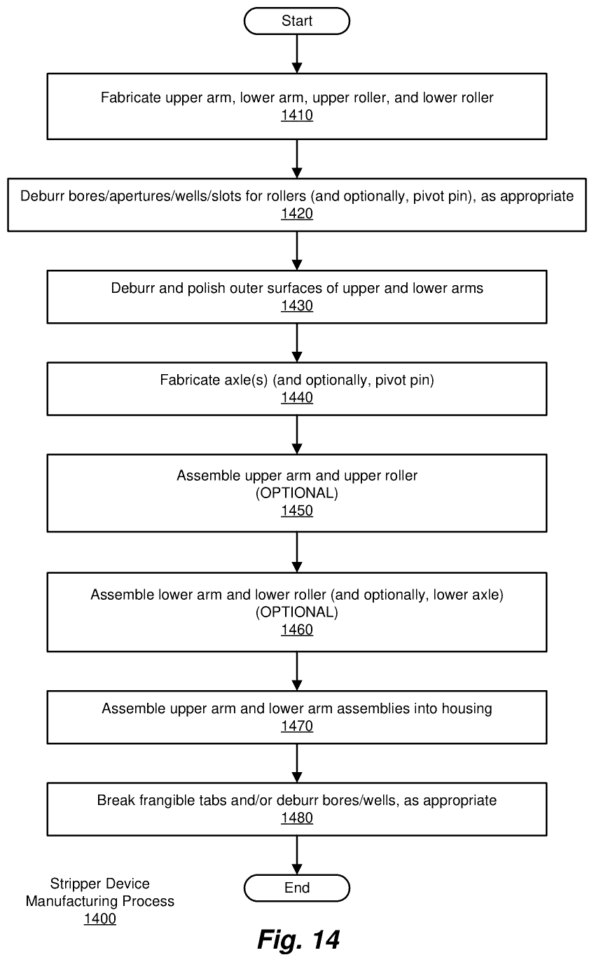

Following initialization, stripper device manufacturing process 1300 proceeds to load design into 3D printing system (1320). In loading the device's design into 3D printing system (1320), a digital model of a surgical drain stripper device such as surgical drain stripper device 100, including the various design elements such as arms, rollers, apertures, and wells, is uploaded to the printer. This model may include specific parameters for structural features such as frangible tabs to facilitate 3D printing and assembly, as well as ensuring alignment of the components of the surgical drain stripper device during manufacturing. It is also to be appreciated that, while the operations described in connection with FIG. 13 (as well as FIG. 14, and generally) are described as being performed in a certain order, such need not be the case, as noted elsewhere herein. That being said, certain operations necessarily follow other operations (e.g., if the 3D printing material used requires curing, the curing operation will necessarily follow the forming step performed, of course).

Next, stripper device manufacturing process 1300 moves to execute the 3D printing of the stripper device (1330). During execution of the 3D printing of the stripper device (1330), the printing system fabricates the main structural components of the surgical drain stripper device in a layer-by-layer fashion. This operation ensures that the housing, arms, and internal features are constructed with in agreement with each other's dimensions, adhering to the tolerances specified in the design. The process may employ support structures or frangible tabs for stability during fabrication.

Optionally, stripper device manufacturing process 1300 includes a load and execute operation to effect 3D printing of rollers and/or other components (1340), depending on the embodiment being produced. This operation allows for the fabrication of additional components, such as rollers, separately from the main structure. These components may include upper roller 150 and lower roller 170, ensuring they are produced with the correct geometry and material properties for their functional roles in compressing drainage tube 108. In such an embodiment, the housing of the surgical drain stripper device is printed, and then the upper and lower rollers printed, followed by insertion of the upper and lower rollers into the housing of the surgical drain stripper device, for example.

Stripper device manufacturing process 1300 then advances to the curing of the 3D printing material (1350). Curing of the 3D printing material involves treating the printed parts to enhance their mechanical properties and stability. This curing process may include thermal curing in an oven or exposure to ultraviolet light, depending on the material used. The curing operation ensures durability and precision of the fabricated parts.

Subsequent to curing, stripper device manufacturing process 1300 proceeds to the breaking of frangible tabs and deburring of bores/apertures/wells (1360). In performing these actions, the temporary supports or tabs that held components in place during printing are removed. This operation also includes deburring internal features such as axle bores and roller wells to eliminate rough edges that could impede the movement or alignment of parts. As will be appreciated in light of the present disclosure, one or more of the frangible tabs fabricated as part of stripper device manufacturing process 1300 can be left in place for transport and delivery to a given customer, and the customer relied on to break such frangible tabs and sufficiently smooth any remaining rough edges (e.g., as by rolling rollers in their roller bores, smoothing snap-in components prior to final assembly, and so on).

Finally, stripper device manufacturing process 1300 concludes with deburring and polishing the outer sues of the stripper device (1370). In performing such deburring and polishing, the external surfaces of the surgical drain stripper device are finished to remove any residual roughness or imperfections from the fabrication process. This operation ensures a smooth finish for both aesthetic appeal and ease of handling during use.

After completing the deburring and polishing of the outer surfaces of the stripper device, stripper device manufacturing process 1300 concludes, yielding a fully fabricated surgical drain stripper device, ready for any remaining assembly operations (e.g., snapping in rollers) and clinical application. Each operation in the process ensures that the final product meets the required standards for functionality, durability, and usability in maintaining surgical drainage systems.

FIG. 14 is a flow diagram illustrating an example of another stripper device manufacturing process, according to embodiments of methods and systems such as those disclosed herein. FIG. 14 thus depicts a stripper device manufacturing process 1400. Stripper device manufacturing process 1400 begins, in the example depicted in FIG. 14, with fabricating upper arm, lower arm, upper roller, and lower roller (1410). Fabricating the various structural and functional components of a surgical drain stripper device such as surgical drain stripper device 1100 (e.g., upper arm 1110, lower arm 1115, upper roller 1150, and lower roller 1170 of surgical drain stripper device 1100, as described in connection with FIGS. 11A and 11B), can be accomplished using a variety of manufacturing techniques, including one or more of deposition fabrication (e.g., as by 3D printing techniques, laser sintering, or other such techniques), forming/molding (e.g., as by injection molding), machining (e.g., as by computational numeric control (CNC) machining), or other such manufacturing techniques. This operation ensures that each component adheres to the design specifications for size, shape, and material properties. When using 3D printing techniques, this operation includes initializing the 3D printing system, loading the designs for the surgical drain stripper device into the 3D printing system, executing 3D printing of the aforementioned surgical drain stripper device/its components, and curing the fabricate pieces. As noted elsewhere herein, the 3D printing of the upper and lower arms can include printing the upper and lower rollers therein, respectively.

Stripper device manufacturing process 1400 then proceeds with the deburring of inner surfaces such as bores, apertures, wells, slots of rollers and/or other such load-bearing surfaces, as appropriate (1420). During this operation, the internal features such as bores, apertures, and wells for rollers and the optional pivot pin (e.g., lower axle bores 1186a and 1186b or upper spindle wells 1170a and 1170b) are smoothed to remove any rough edges or imperfections resulting from the fabrication process. This step ensures that rollers and axles fit securely and rotate freely without excessive friction or misalignment.

Next, the process of stripper device manufacturing process 1400 proceeds with the deburring and polishing of outer surfaces of upper and lower arms (1430). This operation involves smoothing and finishing the external surfaces of the housing (e.g., upper arm 1110 and lower arm 1115) to remove residual imperfections from fabrication. This step enhances the aesthetics and usability of surgical drain stripper device 1100, ensuring that it is comfortable for users to handle and free of sharp edges.

In the embodiment shown in FIG. 4, following surface finishing, one or more axles (and optionally, pivot pin) are fabricated (1440). Fabricating an axle involves creating a lower axle such as lower axle 1184 to specifications that ensure compatibility with the lower roller (e.g., lower roller 1170) and the bores of the lower roller shroud. In embodiments where a through-hole pivot pin is employed (e.g., through-hole pivot pin 1190), this operation also includes fabricating through-hole pivot pin 1190 to allow proper hinging and alignment of the upper and lower arms (e.g., upper arm 1110 and lower arm 1115). As will be appreciated in light of the present disclosure, such a pivot pin can be a through-hole pivot pin or in multiple pieces (e.g., one to a side). As is also to be appreciated, such embodiments will have one or more spring mechanisms (e.g., one or more springs, flaps of housing material that force the arms apart, flexible tabs on either side of the rear aperture(s) that keep the arms apart, or the like), such that the arms are spring-loaded in some fashion.

The upper arm and upper roller are then assembled (1450). As will be appreciated in light of the present disclosure, this is an optional step, as it is dependent on the design of the upper roller and arm. In embodiments where the upper roller (e.g., upper roller 1150) is fabricated separately, this operation can involve mounting the upper roller (e.g., upper roller 1150) in the upper arm (e.g., upper arm 1110). The upper roller is positioned within the upper roller shroud, and its upper spindles (e.g., upper spindles 1175a and 1175b) are seated into the corresponding ones of the upper spindle wells (e.g., upper spindle wells 1170a and 1170b). This assembly operation can include ensuring that the upper roller rotates smoothly within the upper arm.

Next, stripper device manufacturing process 1400 proceeds with assembling the lower arm and lower roller (and optionally, lower axle) (1460). In this operation, the lower roller (e.g., lower roller 1170) is mounted into the lower arm (e.g., lower arm 1115). A lower axle (e.g., lower axle 1184) is passed through the lower axle bores (e.g., lower axle bores 1186a and 1186b) in the lower roller and lower roller shroud, and rotational stability is confirmed. This assembly enables the lower roller to function effectively during compression of the drainage tube.

Subsequent to the assembly of individual components, the process includes assembling upper arm and lower arm assemblies into housing (1470). This operation involves connecting the upper arm (e.g., upper arm 1110) and lower arm (e.g., lower arm 1115) using a through-hole pivot pin such as through-hole pivot pin 1190. In embodiments utilizing springs (e.g., first spring 1195a and second spring 1195b), these are installed on through-hole pivot pin 1190 to maintain the arms in an open position and provide resistive force during operation. This assembly ensures the proper alignment and functionality of surgical drain stripper device 1100.o

Following housing assembly, one or more of any frangible tabs that were fabricated can then be broken (depending on whether any are to be left intact for shipping, for example) and/or deburring bores and wells, as appropriate (1480). This operation removes temporary supports or tabs that were included during the fabrication process to stabilize components. It also smooths internal features, such as those associated with upper spindles and lower axles, ensuring that all components operate smoothly and without interference.

Finally, stripper device manufacturing process 1400 concludes with completing the deburring and polishing of outer surfaces of upper and lower arms. This final polishing operation ensures that the external surfaces of surgical drain stripper device 1100 are free of residual imperfections and visually appealing, preparing the device for clinical use.

At the completion of all operations in stripper device manufacturing process 1400, the surgical drain stripper device is fully fabricated, assembled, and ready for deployment in clinical settings to maintain surgical drainage systems effectively. Each operation ensures the durability, functionality, and usability of the device.

### Examples of Materials

As noted earlier, a surgical drain tube can be used in a variety of scenarios, and facilitate effective fluid management and promote optimal postoperative recovery in a variety of surgical procedures to. Following extensive tissue manipulation, as commonly encountered in procedures such as abdominoplasty and breast augmentation, fluid accumulation can occur. This fluid, which may consist of blood, serum, or lymph, can impede the healing process and increase the risk of complications like seroma formation.

The placement of surgical drain tubes allows for the controlled evacuation of these fluids. By effectively removing excess fluid, these devices mitigate the risk of hematoma formation, reduce postoperative edema, and create a more favorable environment for wound healing. Furthermore, in procedures involving significant tissue resection or manipulation, such as certain abdominal surgeries or joint replacements, drain tubes can be employed to minimize the risk of fluid accumulation and its associated complications. Such an approach enhances patient outcomes by facilitating a more efficient healing process and reducing the potential for postoperative complications.

Surgical drain tubes are typically crafted from biocompatible materials that provide the necessary flexibility, durability, and resistance to kinking or collapse, ensuring their functionality in various medical applications. Among the most commonly used materials is silicone, valued for its exceptional biocompatibility, pliability, and resistance to degradation. Its transparency also allows medical professionals to monitor fluid flow and identify potential blockages, making it a preferred choice for many closed drainage systems.

Polyvinyl chloride (PVC) is another widely utilized material, offering a balance between flexibility, durability, and cost-efficiency. Although slightly more rigid than silicone, PVC is a reliable choice for temporary drains where affordability is a consideration. Similarly, polyurethane is frequently employed for its superior tensile strength, elasticity, and resistance to tearing or chemical degradation, making it an excellent option for situations requiring long-term durability.

Latex rubber, though less commonly used due to the risk of allergic reactions, remains a viable option in certain applications, such as simple Penrose drains, due to its elasticity and affordability. Additionally, thermoplastic elastomers (TPEs) are gaining attention as an alternative material, combining the flexibility of rubber with the processability of plastics. These materials are highly customizable, offering promising advancements in surgical drain technology.

Such materials are chosen based on the specific demands of the surgical drain system, including its biocompatibility, ease of handling, and performance under varying medical conditions. Among these, silicone stands out as a preferred material for its softness and suitability for long-term applications, particularly in sensitive clinical scenarios. As also described earlier, drain tubes, in general, need to be cleared with some frequency, a need that is efficiently, effectively addressed through the use of a surgical drain tube stripper according to embodiments such as those described herein. As noted in this regard, 3D printing can be used to fabricate a surgical drain tube stripper, in its entirety or by fabricating one or more of its component parts.

3D printing, also known as additive manufacturing, leverages a diverse array of materials to fabricate three-dimensional objects. Thermoplastics, such as acrylonitrile butadiene styrene (ABS) and polylactic acid (PLA), are among the most prevalent materials due to their ease of use, affordability, and versatility. ABS exhibits high impact resistance and durability, making it suitable for robust applications, while PLA is derived from renewable resources and offers biodegradability. Another material that can be used to good effect is thermoplastic polyurethane (TPU).

Beyond thermoplastics, the 3D printing processes contemplated by the present disclosure can include a wide spectrum of materials, including metals, ceramics, and other such materials. Metal 3D printing techniques, such as selective laser melting (SLM) and electron beam melting (EBM), enable the fabrication of intricate metal components with high strength and precision. Ceramics, known for their exceptional heat resistance and durability, are utilized in 3D printing for applications in aerospace, automotive, and biomedical sectors.

For a thermoplastic material such as ABS, PLA, or TPU, such materials can provide good results with Shore A hardness values, after curing, as shown in Table 1:

TABLE 1
Example Shore A values.

| Dimension | Example Shore A Values |
|---|---|
| ABS | ~80 to ~95 |
| PLA | ~60 to ~80 |
| TPU | ~60 to ~90 |

Such hardnesses provide a good balance of rigidity and flexibility, while making the surgical drain tube stripper sufficiently rigid and durable.

Using such materials, the dimensions of a surgical drain tube stripper (using surgical drain tube stripper 100 with a Shore D hardness between ~70 and ~90 as an example) can, in general terms, can be as shown in Table 2.

TABLE 2
Example weight band dimensions.

| Dimension | Example Values | |
|---|---|---|
| 3D printed layer thickness | ~0.2 mm to ~0.3 mm | |
| Overall length | ~28 | mm |
| Overall width | ~10 | mm |
| Distance between inner surface of arms (open) | ~11 | mm |
| Distance between inner surface of arms (closed) | ~6 | mm |
| Height of frontispiece of upper arm | ~7 | mm |
| (lower arm stop, from inner surface of | | |
| upper arm) | | |
| Arm thickness | ~4 | mm |
| Shrouds (upper & lower) from spindle centers | ~7 | mm |
| Roller position | approximately | |
| | center of arm | |
| Roller diameter | ~10 | mm |
| Spindle diameter | ~2 | mm |
| Spindle slot width | ~3 | mm |
| Spindle-to-spindle (center) | ~12 | mm |
| Flexible coupling | ~12 | mm tall |
| | ~7 | mm deep |
| Flexion area | ~3 | mm deep |
| | ~2 | mm wide |
| | (@ inner surface) | |
| Housing thickness at flexion area | ~1 mm to ~2 mm | |
| Front aperture | ~4 | mm |
| Rear aperture | ~4 | mm |

Thermoplastics have a number of advantageous characteristics in surgical drain tube strippers such as those described herein, providing effective operation and durability. These benefits stem from their inherent material properties and their compatibility with additive manufacturing processes.
Ease of Processing and Reusability: Thermoplastics are highly suited for 3D printing due to their ability to be melted, reshaped, and solidified without significant degradation. This property facilitates the layer-by-layer construction of intricate components such as arms, rollers, apertures, and wells of a surgical drain tube stripper. Moreover, any excess or failed prints can often be recycled, reducing material waste and lowering production costs.
Customizability and Precision: Thermoplastics can be easily modified to meet specific requirements for mechanical strength, flexibility, and thermal resistance. For example, components like rollers and arms that require rigidity and wear resistance can be made from reinforced or engineering-grade thermoplastics, while parts needing flexibility, such as snap-fit connectors or frangible tabs, can utilize more pliable variants. This adaptability ensures that the finished surgical drain stripper device meets the requisite functional and ergonomic standards.
Biocompatibility and Sterilizability: Many thermoplastics used in medical applications, such as polycarbonate (PC), acrylonitrile butadiene styrene (ABS), and polypropylene (PP), offer excellent biocompatibility, making them safe for contact with surgical environments. Additionally, these materials can withstand sterilization processes like autoclaving or chemical treatments, ensuring that the surgical drain tube stripper remains hygienic and suitable for repeated use in clinical settings.
Lightweight and Durable: Thermoplastics are lightweight yet robust, offering high strength-to-weight ratios. This is particularly advantageous for surgical tools, where ease of handling and durability are critical. A surgical drain tube stripper made from thermoplastic materials is easy for medical staff to maneuver while being resistant to impact, wear, and corrosion.
Cost-Effectiveness and Scalability: Thermoplastics are widely available and cost-effective compared to other materials such as metals or ceramics. Their compatibility with various 3D printing technologies, including fused deposition modeling (FDM) and selective laser sintering (SLS), allows for efficient prototyping and mass production. This scalability supports iterative design processes and reduces time-to-market for new or improved surgical devices.

### An Example Computing and Network Environment

As noted, the 3D printing systems described herein can be implemented using a variety of computer systems and networks. The following illustrates an example configuration of such a computing device. The computing device may include one or more processors, a random access memory (RAM), communication interfaces, a display device, other input/output (I/O) devices (e.g., keyboard, trackball, and the like), and one or more mass storage devices (e.g., optical drive (e.g., CD, DVD, or Blu-ray), disk drive, solid state disk drive, non-volatile memory express (NVME) drive, or the like), configured to communicate with each other, such as via one or more system buses or other suitable connections. While a single system bus is illustrated for ease of understanding, it should be understood that the system buses may include multiple buses, such as a memory device bus, a storage device bus (e.g., serial ATA (SATA) and the like), data buses (e.g., universal serial bus (USB) and the like), video signal buses (e.g., ThunderBolt®, DVI, HDMI, and the like), power buses, or the like.

Such CPUs are hardware devices that may include a single processing unit or a number of processing units, all of which may include single or multiple computing units or multiple cores. Such a CPU may include a graphics processing unit (GPU) that is integrated into the CPU or the GPU may be a separate processor device. The CPU may be implemented as one or more microprocessors, microcomputers, microcontrollers, digital signal processors, central processing units, graphics processing units, state machines, logic circuitries, and/or any devices that manipulate signals based on operational instructions. Among other capabilities, the CPU may be configured to fetch and execute computer-readable instructions stored in a memory, mass storage device, or other computer-readable storage media.

Memory and mass storage devices are examples of computer storage media (e.g., memory storage devices) for storing instructions that can be executed by the processors 502 to perform the various functions described herein. For example, memory can include both volatile memory and non-volatile memory (e.g., RAM, ROM, or the like) devices. Further, mass storage devices may include hard disk drives, solid-state drives, removable media, including external and removable drives, memory cards, flash memory, floppy disks, optical disks (e.g., CD, DVD, Blu-ray), a storage array, a network attached storage, a storage area network, or the like. Both memory and mass storage devices may be collectively referred to as memory or computer storage media herein and may be any type of non-transitory media capable of storing computer-readable, processor-executable program instructions as computer program code that can be executed by the processors as a particular machine configured for carrying out the operations and functions described in the implementations herein.

The computing device may include one or more communication interfaces for exchanging data via a network. The communication interfaces can facilitate communications within a wide variety of networks and protocol types, including wired networks (e.g., Ethernet, DOCSIS, DSL, Fiber, USB, etc.) and wireless networks (e.g., WLAN, GSM, CDMA, 802.11, Bluetooth, Wireless USB, ZigBee, cellular, satellite, etc.), the Internet and the like. Communication interfaces can also provide communication with external storage, such as a storage array, network attached storage, storage area network, cloud storage, or the like.

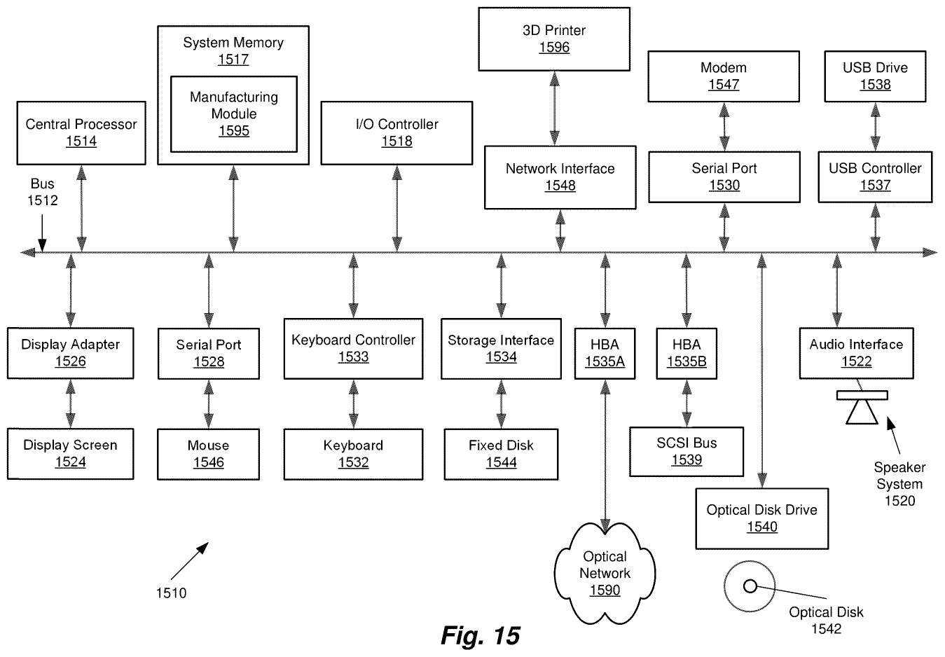

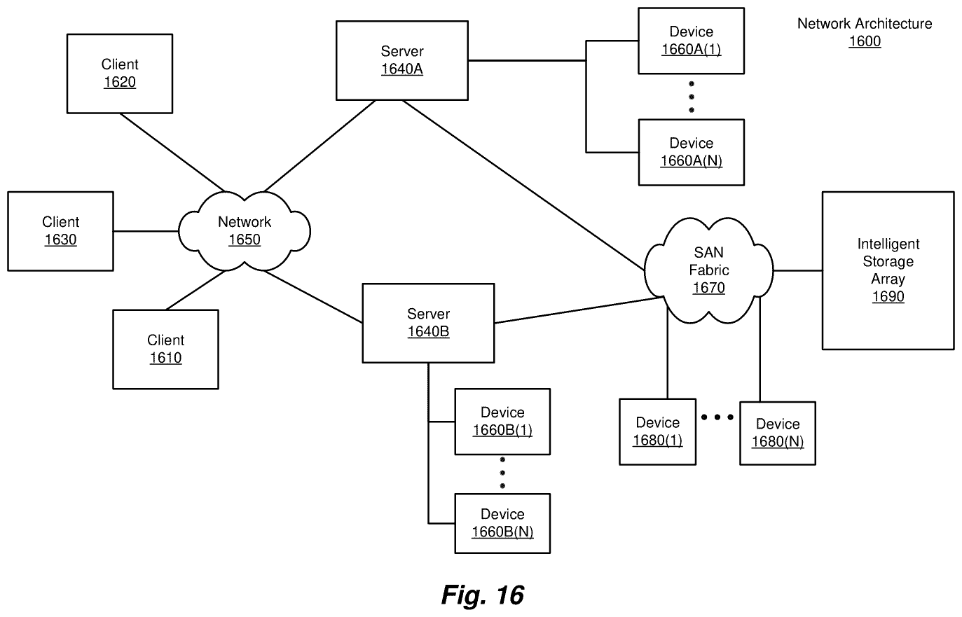

The display device may be used for displaying content (e.g., information and images) to users. Other I/O devices may be devices that receive various inputs from a user and provide various outputs to the user, and may include a keyboard, a touchpad, a mouse, a printer, audio input/output devices, and so forth. The computer storage media, such as memory 504 and mass storage devices, may be used to store software and data, such as, for example, an operating system, one or more drivers (e.g., including a video driver for a display such as display 150), one or more applications, and data. Examples of such computing and network environments are described below with reference to FIGS. 15 and 16.

FIG. 15 depicts a block diagram of a computer system 1510 suitable for implementing aspects of the systems described herein. Computer system 1510 includes a bus 1512 which interconnects major subsystems of computer system 1510, such as a central processor 1514, a system memory 1517 (typically RAM, but which may also include ROM, flash RAM, or the like), an input/output controller 1518, an external audio device, such as a speaker system 1520 via an audio output interface 1522, an external device, such as a display screen 1524 via display adapter 1526, serial ports 1528 and 1530, a keyboard 1532 (interfaced with a keyboard controller 1533), a storage interface 1534, a USB controller 1537 operative to receive a USB drive 1538, a host bus adapter (HBA) interface card 1535A operative to connect with a optical network 1590, a host bus adapter (HBA) interface card 1535B operative to connect to a SCSI bus 1539, and an optical disk drive 1540 operative to receive an optical disk 1542. Also included are a mouse 1546 (or other point-and-click device, coupled to bus 1512 via serial port 1528), a modem 1547 (coupled to bus 1512 via serial port 1530), and a network interface 1548 (coupled directly to bus 1512).

Bus 1512 allows data communication between central processor 1514 and system memory 1517, which may include read-only memory (ROM) or flash memory (neither shown), and random access memory (RAM) (not shown), as previously noted. RAM is generally the main memory into which the operating system and application programs are loaded. The ROM or flash memory can contain, among other code, the Basic Input-Output System (BIOS) which controls basic hardware operation such as the interaction with peripheral components. Applications resident with computer system 1510 are generally stored on and accessed from a computer-readable storage medium, such as a hard disk drive (e.g., fixed disk 1544), an optical drive (e.g., optical drive 1540), a universal serial bus (USB) controller 1537, or other computer-readable storage medium.

Storage interface 1534, as with the other storage interfaces of computer system 1510, can connect to a standard computer-readable medium for storage and/or retrieval of information, such as a fixed disk drive 1544. Fixed disk drive 1544 may be a part of computer system 1510 or may be separate and accessed through other interface systems. Modem 1547 may provide a direct connection to a remote server via a telephone link or to the Internet via an internet service provider (ISP). Network interface 1548 may provide a direct connection to a remote server via a direct network link to the Internet via a POP (point of presence). Network interface 1548 may provide such connection using wireless techniques, including digital cellular telephone connection, Cellular Digital Packet Data (CDPD) connection, digital satellite data connection or the like. Also depicted as part of computer system 1510 is a manufacturing module 1595, which is resident in system memory 1517 and provides functionality and operations that allow for the design, fabrication, and/or other related operations with respect to manufacturing a drain tube stripper device such as that described herein (e.g., produced by way of a 3D printer 1596 communicatively coupled thereto by way of network interface 1548 and a network such as that shown in FIG. 16).

In certain embodiments, manufacturing module 1595 is executed by central processor 1514 of computer system 1510 and operates as an intermediary system that translates a digital 3D model of the surgical drain stripper device and/or components thereof into instructions for a 3D printer such as 3D printer 1596. In certain embodiments, the process can begin with importing a digital model, typically in formats such as STL (stereolithography), OBJ (object file), or AMF (additive manufacturing file), into the software. Manufacturing module 1595 processes this model by slicing the model into numerous horizontal layers, a procedure known as slicing. Each layer is defined by a set of instructions that dictate the movement of the printer's nozzle (or laser or other printing mechanism), the extrusion or curing of material, and any support structures required for overhanging parts. These instructions are encoded in G-code, for example, a language that the printer interprets to execute the physical fabrication.

Once the model is sliced, manufacturing module 1595 allows the fabricator to configure printing parameters such as layer height, print speed, infill density, and material type. These settings optimize the balance between the resolution, mechanical properties, and speed of the print. Additionally, manufacturing module 1595 may feature tools for detecting and repairing errors in the model, such as gaps or non-manifold geometry, ensuring successful fabrication. Advanced modules often provide simulation capabilities, enabling users to visualize how the printer will execute the job and identify potential issues before starting the print.

After configuration, manufacturing module 1595 sends the G-code instructions to 3D printer 1596 via network interface 1548, for example, which can be a wired connection, wireless network, or memory device. Throughout the printing process, manufacturing module 1595 can monitor the status of 3D printer 1596, providing real-time feedback on parameters like temperature, extrusion rate, and print progress. In some systems, manufacturing module 1595 can dynamically adjust settings to account for anomalies, enhancing reliability. By integrating slicing, parameter control, and monitoring, a manufacturing module such as manufacturing module 1595 serves as a link between the stripper device's digital design and its physical fabrication, facilitating accurate and efficient production of complex geometries.

Many other devices or subsystems (not shown) may be connected in a similar manner (e.g., document scanners, digital cameras and so on). Conversely, all of the devices shown in FIG. 15 need not be present to practice the systems described herein. The devices and subsystems can be interconnected in different ways from that shown in FIG. 15. The operation of a computer system such as that shown in FIG. 15 will be readily understood in light of the present disclosure. Code to implement portions of the systems described herein can be stored in computer-readable storage media such as one or more of system memory 1517, fixed disk 1544, optical disk 1542, or USB drive 1538. The operating system provided on computer system 1510 may be WINDOWS, UNIX, LINUX, IOS, or other operating system.

Moreover, regarding the signals described herein, those skilled in the art will recognize that a signal can be directly transmitted from a first block to a second block, or a signal can be modified (e.g., amplified, attenuated, delayed, latched, buffered, inverted, filtered, or otherwise modified) between the blocks. Although the signals of the above described embodiment are characterized as transmitted from one block to the next, other embodiments may include modified signals in place of such directly transmitted signals as long as the informational and/or functional aspect of the signal is transmitted between blocks. To some extent, a signal input at a second block can be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

FIG. 16 is a block diagram depicting a network architecture 1600 in which client systems 1610, 1620 and 1630, as well as storage servers 1640A and 1640B (any of which can be implemented using computer system 1610), are coupled to a network 1650. Storage server 1640A is further depicted as having storage devices 1660A(1)-(N) directly attached, and storage server 1640B is depicted with storage devices 1660B(1)-(N) directly attached. Storage servers 1640A and 1640B are also connected to a SAN fabric 1670, although connection to a storage area network is not required for operation. SAN fabric 1670 supports access to storage devices 1680(1)-(N) by storage servers 1640A and 1640B, and so by client systems 1610, 1620 and 1630 via network 1650. An intelligent storage array 1690 is also shown as an example of a specific storage device accessible via SAN fabric 1670.

With reference to computer system 1510, modem 1547, network interface 1548 or some other method can be used to provide connectivity from each of client computer systems 1610, 1620 and 1630 to network 1650. Client systems 1610, 1620 and 1630 are able to access information on storage server 1640A or 1640B using, for example, a web browser or other client software (not shown). Such a client allows client systems 1610, 1620 and 1630 to access data hosted by storage server 1640A or 1640B or one of storage devices 1660A(1)-(N), 1660B(1)-(N), 1680(1)-(N) or intelligent storage array 1690. FIG. 16 depicts the use of a network such as the Internet for exchanging data, but the systems described herein are not limited to the Internet or any particular network-based environment.

### Other Embodiments

The example methods, systems, and devices described herein are well adapted to attain the advantages mentioned as well as others inherent therein. While such systems have been depicted, described, and are defined by reference to particular descriptions, such references do not imply a limitation on the claims, and no such limitation is to be inferred. The systems described herein are capable of considerable modification, alteration, and equivalents in form and function, as will occur to those ordinarily skilled in the pertinent arts in considering the present disclosure. The depicted and described embodiments are examples only, and are in no way exhaustive of the scope of the claims.

Furthermore, this disclosure provides various example implementations, as described and as illustrated in the drawings. However, this disclosure is not limited to the implementations described and illustrated herein, but can extend to other implementations, as would be known or as would become known to those skilled in the art. Reference in the specification to “one implementation,” “this implementation,” “these implementations” or “some implementations” means that a particular feature, structure, or characteristic described is included in at least one implementation, and the appearances of these phrases in various places in the specification are not necessarily all referring to the same implementation. As such, the various embodiments of the systems described herein via the use of block diagrams, flowcharts, and examples. It will be understood by those within the art that each block diagram component, flowchart step, operation and/or component illustrated by the use of examples can be implemented (individually and/or collectively) in a wide range manners and/or any combination thereof.

In light of the foregoing, it will be appreciated that the foregoing descriptions are intended to be illustrative and should not be taken to be limiting. As will be appreciated in light of the present disclosure, other embodiments are possible. Those skilled in the art will readily implement the steps necessary to provide the structures and the methods disclosed herein, and will understand that the process parameters and sequence of steps are given by way of example only and can be varied to achieve the desired structure as well as modifications that are within the scope of the claims. Variations and modifications of the embodiments disclosed herein can be made based on the description set forth herein, without departing from the scope of the claims, giving full cognizance to equivalents thereto in all respects.

Although the present invention has been described in connection with several embodiments, the invention is not intended to be limited to the specific forms set forth herein. On the contrary, it is intended to cover such alternatives, modifications, and equivalents as can be reasonably included within the scope of the invention as defined by the appended claims.

What is claimed is:

1. An apparatus comprising:
a first arm, wherein
the first arm comprises
a first end of the first arm, and
a second of the first arm;
a first compression mechanism, mechanically coupled to the first arm;
a second arm comprising a first end and a second end; and
the second arm comprises
a first end of the second arm, and
a second end of the second arm;
a second compression mechanism, mechanically coupled to the second arm, wherein
the first arm and the second arm are mechanically coupled to one another at the first end of the first arm and the first end of the second arm,
the first compression mechanism is disposed in relation to the first end of the first arm and the second end of the first arm, and the second compression mechanism is disposed in relation to the first end of the second arm and the second end of the second arm, such that, when the second end of the first arm and the second end of the second arm are brought sufficiently close to one another, the first compression mechanism and the second compression mechanism are configured to apply compressive pressure to a surgical drain tube positioned therebetween, and
the first compression mechanism and the second compression mechanism are configured to allow the apparatus to be moved along the surgical drain tube while applying the compressive pressure to the surgical drain tube.

2. The apparatus of claim 1, wherein
the first compression mechanism and the second compression mechanism are configured to allow the apparatus to be moved along the surgical drain tube while applying the pressure to the surgical drain tube by virtue of
at least one of the first compression mechanism and the second compression mechanism being a first roller, and
the first compression mechanism and the second compression mechanism apply the pressure to the surgical drain tube by virtue of moving in opposition to one another against the surgical drain tube.

3. The apparatus of claim 2, wherein
the first compression mechanism is the first roller,
the second compression mechanism is a second roller.

4. The apparatus of claim 3, further comprising:
a coupling, wherein
an axis of the first roller is substantially parallel to a jaw axis of the device,
an axis of the second roller is substantially parallel to the jaw axis of the device,
the coupling comprises an aperture, and
the aperture is disposed in the coupling such that the coupling permits the surgical drain tube to be threaded through the coupling and between the first compression mechanism and the second compression mechanism.

5. The apparatus of claim 4, wherein
the apparatus is configured to be moved towards the distal end of the surgical drain tube by virtue of the apparatus being configured to be oriented such that
the second end of the first arm and the second end of the second arm are proximal to a proximal end of the surgical drain tube that is to be inserted into a patient, along the surgical drain tube, and
the coupling is distal to the proximal end of the surgical drain tube that is to be inserted into the patient, along the surgical drain tube.

6. The apparatus of claim 3, wherein
an axis of the first roller is substantially parallel to a jaw axis of the device, and
an axis of the second roller is substantially parallel to the jaw axis of the device.

7. The apparatus of claim 2, wherein
at least another of the first compression mechanism and the second compression mechanism is a low-friction pad,
a friction between the low-friction pad and the surgical drain tube causes the surgical drain tube to experience a first force
the first force is less than a force that would result in extraction of the surgical drain tube from a patient into whom the surgical drain tube had been inserted.

8. The apparatus of claim 1, wherein
the first compression mechanism and the second compression mechanism are configured to allow the apparatus to be moved along the surgical drain tube while applying the pressure to the surgical drain tube by virtue of
the first compression mechanism being a first low-friction pad, and
the second compression mechanism being a second low-friction pad, and
the low-friction pad and the second low-friction pad apply the pressure to the surgical drain tube by virtue of moving in opposition to one another against the surgical drain tube.

9. The apparatus of claim 8, wherein
a first friction between the first compression mechanism and the surgical drain tube causes the surgical drain tube to experience a first force,
a second friction between the second compression mechanism and the surgical drain tube causes the surgical drain tube to experience a second force, and
the first force and the second force are less than a force that would result in extraction of the surgical drain tube from a patient into whom the surgical drain tube had been inserted.

10. The apparatus of claim 1, wherein
the first compression mechanism and the second compression mechanism are configured to apply a sufficient pressure to the surgical drain tube positioned therebetween to collapse the surgical drain tube.

11. The apparatus of claim 1, further comprising:
a coupling, wherein
the coupling is
a flexible coupling that flexibly couples the first end of the first arm and the first end of the second arm, or
a rotating coupling that rotatably couples the first end of the first arm and the first end of the second arm.

12. The apparatus of claim 1, further comprising:
a stop, wherein
the stop limits travel of the first compression mechanism and/or travel of the second compression mechanism towards the second arm and/or the first arm, respectively.

13. The apparatus of claim 12, further comprising:
the stop limits a first force, applied to the surgical drain tube by the first compression mechanism, and a second force, applied to the surgical drain tube by the second compression mechanism.

14. The apparatus of claim 1, wherein
the apparatus is configured to create additional suction in the surgical drain tube, as a result of deformation of the surgical drain tube and elastic recovery of an original shape of the surgical drain tube, and to clear the surgical drain tube by virtue of being configured to break up debris inside surgical drain tube and to force one or more resulting pieces of the debris and/or fluid through the surgical drain tube a direction distally to a patient into which the surgical drain tube is inserted, in response to a pressure applied to the surgical drain tube by the first compression mechanism and the second compression mechanism, and movement of the apparatus along the surgical drain tube in the direction distally to the patient into which the surgical drain tube is inserted.
